(19) United States
(12) Patent Application Publication
(10) Pub. No.: US 2024/0122028 A1
(43) Pub. Date: Apr. 11, 2024

(54) ELECTRONIC DEVICE

(71) Applicant: SEMICONDUCTOR ENERGY LABORATORY CO., LTD., Atsugi-shi, Kanagawa-ken (JP)

(72) Inventors: Takayuki IKEDA, Atsugi, Kanagawa (JP); Hisao IKEDA, Zama, Kanagawa (JP); Tatsuya ONUKI, Atsugi, Kanagawa (JP); Shunpei YAMAZAKI, Setagaya, Tokyo (JP)

(21) Appl. No.: 18/274,810

(22) PCT Filed: Feb. 7, 2022
(86) PCT No.: PCT/IB2022/051022

§ 371 Date: Jul. 28, 2023

(30) Foreign Application Priority Data
Jul. 18, 2021 (JP) 2021-024547

(51) Int. Cl. H10K 59/65; A61B 3/00; A61B 3/12; A61B 3/14; H10K 59/90; H10K 59/95

(52) U.S. Cl. CPC H10K 59/65; A61B 3/005; A61B 3/12; A61B 3/14; H10K 59/90; H10K 59/95; H01L 24/05

(57) ABSTRACT

An object of one embodiment of the present invention is to provide a novel display device or a display system. Another object of one embodiment of the present invention is to provide a display device or a display system which can be manufactured at low cost and can provide various functions to a user. A pixel includes light-emitting elements whose emission colors are different from each other, a light-emitting element IR, a light-receiving element PS, and an infrared light sensor IRS. An image of a fundus of an eye is captured using the light-emitting element emitting an infrared light as a light source, and imaging is performed by the light-receiving element IRS. A substrate of a display panel is manufactured using a single crystal Si substrate capable of microfabrication and higher integration.

### TECHNICAL FIELD

The present invention relates to a semiconductor device and a manufacturing method thereof. In particular, one embodiment of the present invention relates to an electronic device utilizing organic electroluminescence (hereinafter also referred to as EL) phenomenon and a display system.

Note that in this specification, a semiconductor device refers to any device that can function by utilizing semiconductor characteristics, and an electro-optical device, a semiconductor circuit, and an electronic device are all semiconductor devices.

### BACKGROUND ART

A variety of display devices ranging from a large display device typified by a television receiver to a small display device typified by a wristwatch has been spread in a market. In recent years, information terminal devices typified by mobile phones typified by smartphones, tablet information terminals, and laptop PCs (personal computers) have been widely used. A head-mounted type or glasses-type display device (Patent Document 1) is also developed.

A light-emitting element utilizing an EL phenomenon is known. This light-emitting element is a self-luminous type; accordingly, high contrast and high-speed response to an input signal are achieved. Furthermore, a display device to which this light-emitting element is applied and which consumes less power and which is manufactured in a simple process and is easily adapted to the increase in definition and the size of a substrate is known (Patent Document 2).

### REFERENCES

### Patent Documents

[Patent Document 1] Japanese Published Patent Application No. 2016-110117
[Patent Document 2] Japanese Published Patent Application No. 2011-238908

### SUMMARY OF THE INVENTION

### Problems to be Solved by the Invention

An object of one embodiment of the present invention is to provide a novel display device or a display system. Another object of one embodiment of the present invention is to provide a display device or a display system which can be manufactured at low cost and can provide various functions to a user. Another object of one embodiment of the present invention is to provide a display device or a display system that can obtain information on a user's eye.

### Means for Solving the Problems

A structure disclosed in this specification is an electronic device worn on a head of a user, including a display device including a transistor, a light-emitting element, and a light-receiving element in the same substrate or over the same substrate. The display device has a function of displaying an image and a function of capturing an image of a fundus of an eye of the user.

In the above structure, the light-emitting element is an organic light-emitting element that emits visible light. Color of light emitted from the organic light-emitting element is red, green, blue, or white.

In the above structure, the light-emitting element is an organic light-emitting element that emits nonvisible light. An example is an organic light-emitting element that emits infrared light.

In each of the above structures, single crystal silicon is used for a semiconductor layer of the transistor. In the case where single crystal silicon is used for the semiconductor layer of the transistor, a low-resistance region is formed by doping a single crystal Si substrate with an n-type or p-type impurity element by a known method.

In each of the above structures, an oxide semiconductor is used for the semiconductor layer of the transistor. In the case where the semiconductor layer of the transistor is an oxide semiconductor, deposition is performed over a substrate by a sputtering method.

In each of the above structures, the electronic device includes a light-emitting element and a light-receiving element, and makes diagnosis with an image of the fundus of the eye of the user obtained by capturing an image of the user's eye.

In this specification, a device formed using a metal mask or a fine metal mask (FMM) may be referred to as a device having a metal mask (MM) structure. In this specification, a device formed without using a metal mask or an FMM may be referred to as a device having a metal maskless (MML) structure.

In this specification, a structure in which light-emitting layers in light-emitting devices of different colors (here, blue (B), green (G), and red (R)) are separately formed or separately patterned may be referred to as an SBS (Side By Side) structure. In this specification, a light-emitting device capable of emitting white light may be referred to as a white-light-emitting device. Note that a combination of white light-emitting devices and coloring layers (e.g., color filters) achieves a light-emitting apparatus of full-color display.

Structures of light-emitting devices can be classified roughly into a single structure and a tandem structure. A device having a single structure includes one light-emitting unit between a pair of electrodes, and the light-emitting unit preferably includes one or more light-emitting layers. To obtain white light emission, two or more of light-emitting layers are selected such that their emission colors are complementary to each other. For example, when an emission color of a first light-emitting layer and an emission color of a second light-emitting layer are complementary colors, the light-emitting device can be configured to emit white light as a whole. The same applies to a light-emitting device including three or more light-emitting layers.

A device having a tandem structure includes two or more light-emitting units between a pair of electrodes, and each light-emitting unit preferably includes one or more light-emitting layers. To obtain white light emission, the structure is made so that light from light-emitting layers of the plurality of light-emitting units can be combined to be white light. Note that a structure for obtaining white light emission is similar to that in the case of a single structure. In the device having a tandem structure, it is preferable that an intermediate layer such as a charge-generation layer is provided between a plurality of light-emitting units.

When the white-light-emitting device (having a single structure or a tandem structure) and a light-emitting device having an SBS structure are compared to each other, the light-emitting device having an SBS structure can have lower power consumption than the white-light-emitting device. To reduce power consumption, a light-emitting device having an SBS structure is preferably used. Meanwhile, the white-light-emitting device is suitable in terms of lower manufacturing cost or higher manufacturing yield because the manufacturing process of the white-light-emitting device is simpler than that of the light-emitting device having an SBS structure.

### Effect of the Invention

One embodiment of the present invention can provide an electronic device which can precisely recognize information on a user's eye. Another embodiment of the present invention can provide a novel electronic device.

### BRIEF DESCRIPTION OF THE DRAWINGS

FIG. 1A illustrates schematic cross-sectional views of a display device and an eye illustrating one embodiment of the present invention, FIG. 1B is a schematic view illustrating a retina pattern, and FIG. 1C is a perspective view of a display panel.

FIG. 2A is a perspective view of a display panel illustrating one embodiment of the present invention, and FIG. 2B and FIG. 2C are schematic top views each illustrating an example of pixel arrangement.

FIG. 3A is a schematic cross-sectional view illustrating one embodiment of the present invention, and FIG. 3B and FIG. 3C are schematic external views of an electronic device.

FIG. 4A, FIG. 4B, and FIG. 4C are cross-sectional views of a display panel illustrating one embodiment of the present invention.

FIG. 5A, FIG. 5B, and FIG. 5C are schematic diagrams of display panels illustrating one embodiment of the present invention.

FIG. 6 is a perspective view of an electronic device illustrating one embodiment of the present invention.

FIG. 7A is a top view illustrating an arrangement example of a pixel illustrating one embodiment of the present invention, and FIG. 7B is a schematic cross-sectional view of a display panel.

FIG. 8 is a schematic top view illustrating one embodiment of the present invention.

FIG. 9 is a cross-sectional view illustrating a structure example of a semiconductor device.

FIG. 10 is a cross-sectional view illustrating a structure example of a semiconductor device.

FIG. 11 is a cross-sectional view illustrating a structure example of a semiconductor device.

FIG. 12 is a cross-sectional view illustrating a structure example of a semiconductor device.

FIG. 13 is a cross-sectional view illustrating a structure example of a semiconductor device.

FIG. 14 is a cross-sectional view illustrating a structure example of a semiconductor device.

FIG. 15A to FIG. 15D are diagrams illustrating structure examples of a light-emitting element.

FIG. 16A to FIG. 16D are diagrams illustrating structure examples of a display device.

FIG. 17A to FIG. 17D are diagrams each illustrating a structure example of a display device.

FIG. 18A is a top view illustrating a structure example of a transistor. FIG. 18B and FIG. 18C are cross-sectional views illustrating the structure example of a transistor.

FIG. 19A is a diagram showing classifications of crystal structures of IGZO. FIG. 19B is a graph showing an XRD spectrum of a CAAC-IGZO film. FIG. 19C is an image showing a nanobeam electron diffraction pattern of a CAAC-IGZO film.

### MODE FOR CARRYING OUT THE INVENTION

Hereinafter, embodiments of the present invention will be described in detail with reference to the drawings. Note that the present invention is not limited to the following description, and it is readily understood by those skilled in the art that modes and details of the present invention can be modified in various ways. In addition, the present invention should not be construed as being limited to the description of the embodiments below.

Note that in structures of the invention described below, the same portions or portions having similar functions are denoted by the same reference numerals in different drawings, and the description thereof is not repeated. Furthermore, the same hatch pattern is used for the portions having similar functions, and the portions are not especially denoted by reference numerals in some cases.

Note that in each drawing described in this specification, the size, the layer thickness, or the region of each component is exaggerated for clarity in some case. Therefore, they are not limited to the illustrated scale.

In this specification, the ordinal numbers such as “first” or “second” are used in order to avoid confusion among components and do not limit the number.

In this specification, terms for describing arrangement, such as “over”, “under”, “left”, and “right”, are used for convenience in describing a positional relation between components with reference to drawings. The positional relation between components is changed as appropriate in accordance with the direction in which the components are described. Thus, without limitation to terms described in this specification, the description can be changed appropriately depending on the situation.

A transistor is a kind of semiconductor elements and can achieve amplification of current or voltage or switching operation for controlling conduction or non-conduction. An IGFET (Insulated Gate Field Effect Transistor) or a thin film transistor (TFT) is in the category of a transistor in this specification.

In this specification, functions of a source and a drain of a transistor are sometimes switched from each other depending on the polarity of the transistor or the case where the direction of current flow is changed in circuit operation. Therefore, the terms “source” and “drain” can be used interchangeably.

In this specification, the expression “electrically connected” includes the case where components are directly connected to each other and the case where components are connected through an “object having any electric function”. Here, there is no particular limitation on the “object having any electric function” as long as electric signals can be transmitted and received between components that are connected through the object. Thus, even when the expression “electrically connected” is used, there is a case where no physical connection is made and a wiring just extends in an actual circuit. In addition, the expression “directly connected” includes the case where different conductors are connected to each other through a contact. Note that a wiring may be formed of conductors that contain one or more of the same elements or may be formed of conductors that contain different elements.

Furthermore, unless otherwise specified, an off-state current in this specification refers to a drain current of a transistor in an off state (also referred to as a non-conduction state or a cutoff state). Unless otherwise specified, an off state refers to, in an n-channel transistor, a state where voltage V_{gs} between its gate and source is lower than the threshold voltage Vₜₕ (in a p-channel transistor, higher than Vₜₕ).

In this specification, the terms such as “electrode” or “wiring” do not limit the functions of the components. For example, an “electrode” is used as part of a “wiring” in some cases, and vice versa. Furthermore, the term “electrode” or “wiring” also includes the case where a plurality of “electrodes” or wirings” are formed in an integrated manner.

In this specification, the resistance value of a “resistor” is sometimes determined depending on the length of a wiring. Alternatively, the resistance value is sometimes determined by connection to a conductor with resistivity different from that of a conductor used for a wiring. Alternatively, the resistance value is sometimes determined by doping a semiconductor with an impurity.

In this specification, a “terminal” in an electric circuit refers to a portion that inputs or outputs current or voltage or receives or transmits a signal. Accordingly, part of a wiring or an electrode functions as a terminal in some cases.

In this specification, a metal oxide is an oxide of metal in a broad sense. Metal oxides are classified into an oxide insulator, an oxide conductor (including a transparent oxide conductor), and an oxide semiconductor (also simply referred to as an OS). For example, in the case where a metal oxide is used in an active layer of a transistor, the metal oxide is referred to as an oxide semiconductor in some cases. That is, an OS FET can also be called a transistor including a metal oxide or an oxide semiconductor.

### Embodiment 1

In this embodiment, an electronic device of one embodiment of the present invention will be described with reference to drawings.

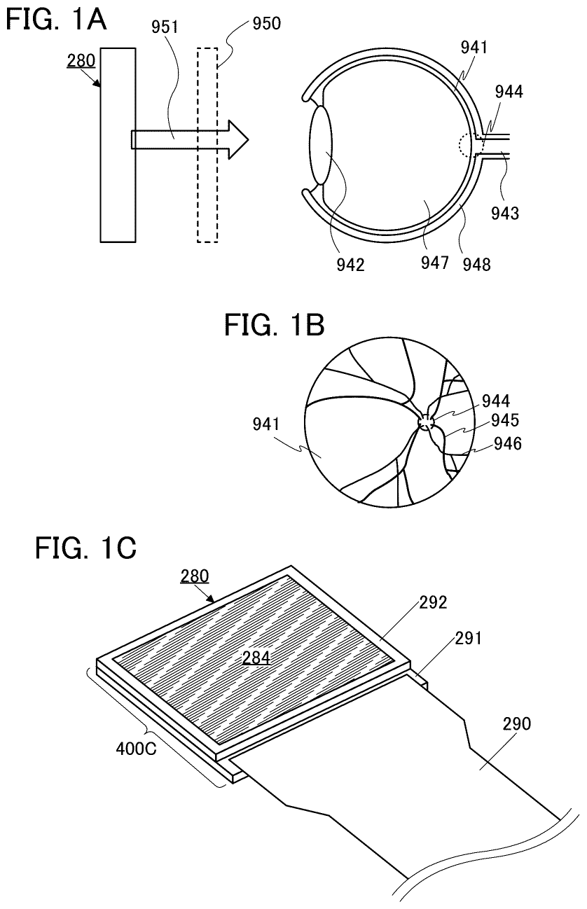

FIG. 1A is a schematic view illustrating a positional relationship between a display panel 280 and a user's eye. The display panel 280 includes a plurality of light-emitting elements and a plurality of light-receiving elements. The light-emitting element of the display panel 280 performs irradiation with emitted light 951, and the light-receiving element receives the light that irradiates the eye through an optical system 950 and is reflected, whereby image capturing of a periphery of the eye, a surface of the eye, and an inner portion (a fundus) of the eye is performed. For example, the display panel 280 illustrated in FIG. 1A includes the light-emitting element and the light-receiving element, and thus can capture an image of the fundus of one's eye through the optical system 950 to obtain image data of a retinal pattern. Note that it is difficult to capture an image of other objects when the focus is adjusted with the optical system 950. For example, when the fundus of the eye is focused on, the periphery of the eye is out of focus and thus an image thereof can hardly be captured.

A user's eye is made up of a crystalline lens 942, a retina 941, an optic nerve 943, a vitreous body 947, a choroid 948, and a cornea. Although there is a pupil between a cornea and a crystalline lens, the cornea and the pupil are not illustrated for simplicity. A ciliary body is a tissue continuous with an iris, and the choroid 948 is a tissue continuous with the ciliary body. Like the diaphragm of a camera, the iris and the pupil adjust the amount of light delivered to the retina 941. It is said that the pattern of the retina 941, what is called the retina pattern basically does not change in one's life, and the retina pattern can be used for personal authentication. Medical examination of an eye is possible even from a remote place with the use of the retina pattern obtained with the display panel 280.

FIG. 1B illustrates an example of an obtained retina pattern of the right eye. An optic disc 944, a vein 945, an artery 946, a macula, or a fovea can be observed in the retina 941. The optic disc 944 is a boundary portion between the optic nerve 943 and the retina 941, and the vein 945 and the artery 946 are positioned to be extended from the optic disc 944. Note that the fundus of the eye is a portion behind the eyeball and generally refers to the retina 941, the vitreous body 947, the choroid 948, and the optic disc 944. The optic disc 944 of the left eye is positioned in the left side of the retina pattern. The retina pattern of the left eye is a mirror-reversed retina pattern of the right eye in FIG. 1B.

To obtain the retina pattern of the fundus of the eye with the use of the light-receiving element in the display panel 280, the pupil needs to be open. Display is changed in the following procedure to capture an image of the fundus of the eye with the pupil open. The display screen of the display panel 280 is gradually darkened so that the user's eye adapts to the dark. Then, the display screen is brightened for a short time of 16.7 ms or less and an image is captured. After that, the brightness of the display screen is gradually returned to the original.

Furthermore, a fatigue level of the user's eye can be detected using the display panel 280. When an image is captured while the display screen is brightened for a certain period of time, an image cannot be captured if the user blinks. In this case, the fatigue level of the eye can be estimated with the use of a system utilizing AI (Artificial Intelligence) from the frequency, blink intervals, or the time when the user closes the eyes, which are obtained by detection of the number or timing of the blinks, or the time when the user closes the eyes.

Moreover, image capturing may be performed a plurality of times while the display screen of the display panel 280 is darkened. Capturing images a plurality of times makes it possible to detect the pulse of the retina blood vessel and furthermore to determine whether the user is in a rest state or in a state of tension with the use of the system utilizing AI. In addition, diagnosis of high blood pressure or diagnosis of diabetes can be made with the use of the system utilizing AI and of a variety of pieces of data obtained with the display panel 280. In the case of employing the system utilizing AI, a control circuit is mounted on the display panel 280. A CPU (Central Processor Unit) or a GPU (Graphics Processing Unit) is included in the control circuit. A chip in which a CPU and a GPU are integrated, an APU (Accelerated Processing Unit), can also be used for the control circuit. AI (an IC incorporating a system (also referred to as an inference chip) may be used. The IC incorporating an AI system is sometimes referred to as a circuit performing neural network computation (a microprocessor).

The movement of an eyeball may be controlled by displaying an eye-catching pattern on the display screen of the display panel 280 while the display screen of the display panel 280 is darkened.

The distance between the display panel 280 and a surface of an eye (for example, a cornea) is preferably less than or equal to approximately 2 cm. The optical system 950 having a short focal distance is provided between the display panel 280 and the eye to achieve this positional relationship.

In the case where an image displayed on a screen is magnified tenfold by the optical system 950, e.g., the case where the size of the display screen of the display panel 280 is set to a diagonal of approximately 1 inch, an image can be captured with the vascular diameter of each of the vein 945 and the artery 946 of the retina smaller than approximately 100 μm although a pitch of a sensor pixel becomes approximately 10.4 μm.

FIG. 1C illustrates a perspective view of the display panel 280. The display panel 280 includes a display device 400C and an FPC 290. The display panel 280 can also be referred to as a system display.

The display panel 280 includes a substrate 291 and a substrate 292. The display panel 280 includes a pixel portion 284. The pixel portion 284 is a region of the display panel 280 where an image is displayed and is a region where light emitted from pixels provided in a pixel portion 284 described later can be seen.

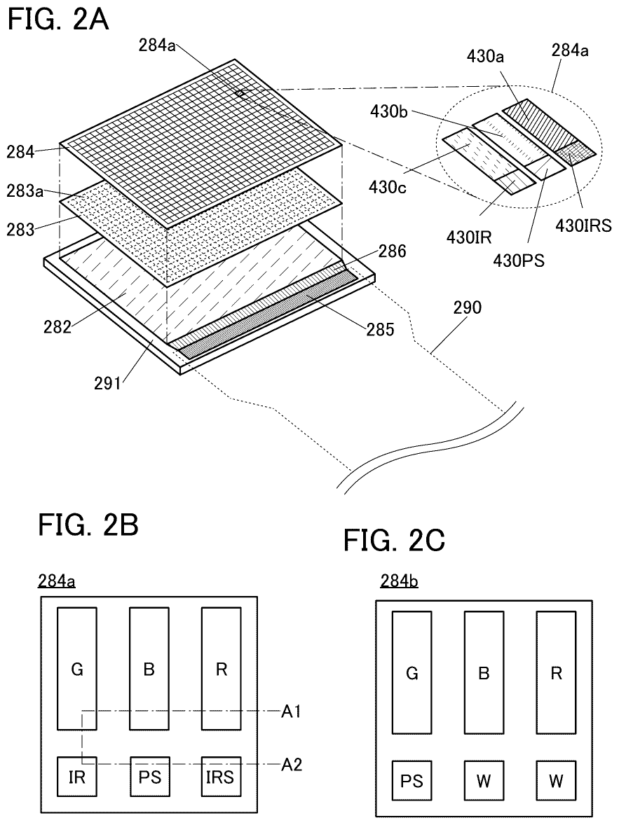

FIG. 2A is a perspective view schematically illustrating a structure on the substrate 291 side. Over the substrate 291, a circuit portion 282, a pixel circuit portion 283 over the circuit portion 282, and the pixel portion 284 over the pixel circuit portion 283 are stacked. In addition, a terminal portion 285 for connection to the FPC 290 is included in a portion not overlapping with the pixel portion 284 over the substrate 291. The terminal portion 285 and the circuit portion 282 are electrically connected to each other through a wiring portion 286 formed of a plurality of wirings.

The pixel portion 284 includes a plurality of pixels 284a arranged periodically. An enlarged view of one pixel 284a is illustrated on the right side in FIG. 2A. The pixel 284a includes light-emitting elements 430a, 430b, and 430c whose emission colors are different from each other, a light-emitting element 43018, a light-receiving element 430PS, and an infrared light sensor 430IRS. Although the light-emitting elements 430a, 430b, and 430c are arranged in a stripe arrangement as illustrated in FIG. 2A, there is no particular limitation on the arrangement. In addition, FIG. 2B illustrates an example of arrangement of the pixel 284a. FIG. 2B illustrates an example in which a green (G) light-emitting element, a blue (B) light-emitting element, a red (R) light-emitting element, the light-emitting element 4301R, the light-receiving element 430PS, and the infrared light sensor 430IRS are arranged.

As each of the light-emitting elements 430a, 430b, and 430c, and the light-emitting element 4301R, an EL element typified by an OLED (Organic Light Emitting Diode) or a QLED (Quantum-dot Light Emitting Diode) is preferably used. As a light-emitting substance contained in the EL element, a substance that emits fluorescence (a fluorescent material), a substance that emits phosphorescence (a phosphorescent material), an inorganic compound (a quantum dot material), and a substance that exhibits thermally activated delayed fluorescence (a thermally activated delayed fluorescent (TADF) material) can be given. An LED typified by a micro-LED (Light Emitting Diode) can be used as the light-emitting element.

The light-emitting element 4301R emits invisible light. Here, an example in which the light-emitting element 4301R emits infrared light IR is illustrated. The light-receiving element 430PS is a photoelectric conversion element having sensitivity to at least infrared light emitted from the light-emitting element 4301R.

The light-receiving element 430PS has sensitivity in a wavelength range of greater than or equal to 700 nm and less than or equal to 900 nm, for example. It is preferable that the light-receiving element 430PS has sensitivity to not only infrared light but also light emitted from each of the light-emitting element 430IR and the light-emitting elements 430a, 430b, and 430c. In the case where the light-receiving element 430PS has sensitivity to visible light and infrared light, the light-receiving element 430PS preferably has sensitivity in a wavelength range of greater than or equal to 500 nm and less than or equal to 1000 nm, a wavelength range of greater than or equal to 500 nm and less than or equal to 950 nm, or a wavelength range of greater than or equal to 500 nm and less than or equal to 900 nm, for example.

As the light-receiving element 430PS, a pn or pin photodiode can be used, for example. The light-receiving element functions as a photoelectric conversion element that detects light incident on the light-receiving element and generates charge. The amount of generated charge in the photoelectric conversion element is determined depending on the amount of incident light. In particular, an organic photodiode (OPD) including a layer containing an organic compound is preferably used as the light-receiving element 430PS. Accordingly, some production facilities and manufacturing apparatuses for manufacturing the first light-emitting element, the second light-emitting element, and the light-receiving element, and some materials that can be used therefor can be used in common; thus, the manufacturing cost can be reduced. Furthermore, the manufacturing processes of the first light-emitting element, the second light-emitting element, and the light-receiving element can be simplified, so that the manufacturing yield can be improved. An organic photodiode, which is easily made thin, lightweight, and large in area and has a high degree of freedom for shape and design, can be used in a variety of display devices.

The pixel circuit portion 283 includes a plurality of pixel circuits 283a arranged periodically.

One pixel circuit 283a is a circuit that controls light emission from three light-emitting elements included in one pixel 284a. One pixel circuit 283a may be provided with three circuits each of which controls light emission of one light-emitting element. For example, the pixel circuit 283a can include at least one selection transistor, one current control transistor (driving transistor), and a capacitor for one light-emitting element. A gate signal is input to a gate of the selection transistor, and a source signal is input to one of a source and a drain of the selection transistor. With such a structure, an active-matrix display device is achieved.

The circuit portion 282 includes a circuit for driving the pixel circuits 283a in the pixel circuit portion 283. For example, one or both of a gate line driver circuit and a source line driver circuit are preferably included. In addition, at least one of an arithmetic circuit, a memory circuit, a control circuit, and a power supply circuit may be included.

The FPC 290 functions as a wiring for supplying a video signal or a power supply potential to the circuit portion 282 from the outside. An IC may be mounted on the FPC 290.

The display panel 280 can have a structure in which one or both of the pixel circuit portion 283 and the circuit portion 282 are stacked below the pixel portion 284; thus, the aperture ratio (the effective display area ratio) of the pixel portion 284 can be significantly high. For example, the aperture ratio of the pixel portion 284 can be greater than or equal to 40% and less than 100%, preferably greater than or equal to 50% and less than or equal to 95%, further preferably greater than or equal to 60% and less than or equal to 95%. Furthermore, the pixels 284a can be arranged extremely densely and thus the pixel portion 284 can have greatly high resolution. For example, the pixels 284a are preferably arranged in the pixel portion 284 with a resolution greater than or equal to 2000 ppi, preferably greater than or equal to 3000 ppi, further preferably greater than or equal to 5000 ppi, still further preferably greater than or equal to 6000 ppi, and less than or equal to 20000 ppi or less than or equal to 30000 ppi.

FIG. 2C illustrates an example of a pixel portion 284b which is different from the pixel portion 284a in FIG. 2B. FIG. 2C is an example in which white (W) light-emitting elements are provided in addition to a green (G) light-emitting element, a blue (B) light-emitting element, a red (R) light-emitting element.

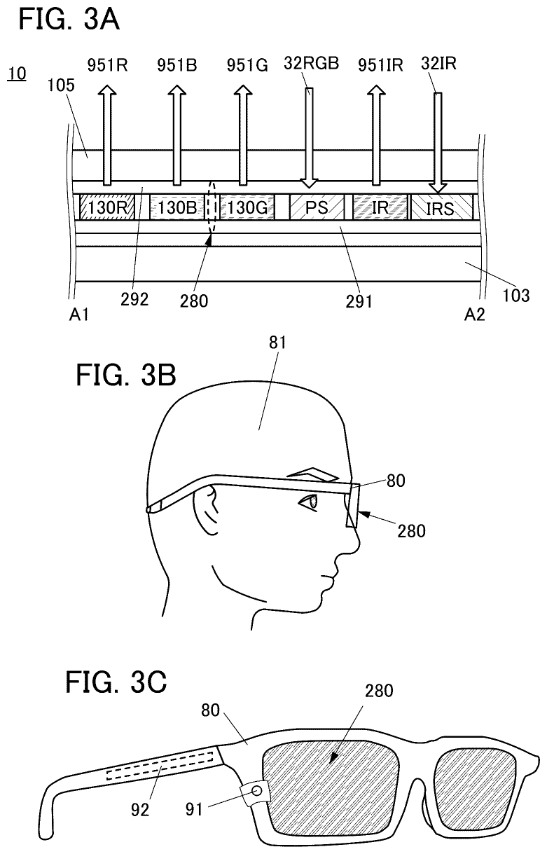

FIG. 3A illustrates an example of a cross-sectional view of an electronic device 80 including a display device of one embodiment of the present invention. FIG. 3C illustrates an example of a glasses-type electronic device 80, and FIG. 3B illustrates an example of the electronic device 80 worn on a user. Although not illustrated, in FIG. 3B, the electronic device 80 includes a secondary battery for driving the display panel 280.

The display panel 280 illustrated in FIG. 3A corresponds to a cross-sectional structure along a dashed-dotted line A1-A2 in FIG. 2B. The electronic device 80 illustrated in FIG. 3A includes the display panel 280 between a housing 103 and a protective member 105. The display panel 280 includes a plurality of light-emitting elements and a plurality of light-receiving elements between the substrate 291 and the substrate 292. Here, FIG. 3A and FIG. 3B are schematic views illustrating a function of the electronic device 80 as a display device and its function of capturing an image of an object (a fundus of an eye of a user 81).

In the electronic device 80, full-color display is performed using a light-emitting element 130R that emits red light 951R, a light-emitting element 130G that emits green light 951G, and a light-emitting element 130B that emits blue light 951B, and fundus image capturing is performed by a light-receiving element IRS using a light-emitting element emitting emitted light 951IR which is infrared light as a light source.

A light-receiving element PS can receive reflected light or external light which is RGB, detect the brightness around an eye, and check whether darkness is ensured around the eye before capturing an image of a fundus of the eye. In the case where the check is not needed, the light-receiving element PS is not necessarily provided.

The optical system is installed in a region overlapping with the light-emitting element emitting the emitted light 951IR which is infrared light and the light-receiving element IRS, so that the fundus of the eye is in focus.

In FIG. 3A, the substrate 291 of the display panel 280 may be formed by using a glass substrate or a plastic substrate to form a switching element over which a light-receiving element or an organic EL element (OEL) electrically connected to the switching element is formed; however, the substrate 291 is preferably manufactured using a single crystal Si substrate capable of microfabrication and higher integration.

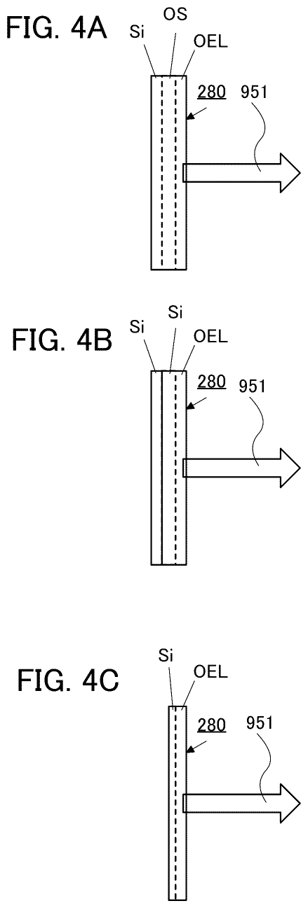

FIG. 4A, FIG. 4B, and FIG. 4C illustrate structural examples of the display panel 280 mounted on the electronic device 80. Note that FIG. 4A, FIG. 4B, and FIG. 4C correspond to FIG. 1A, and the same reference numerals are used for the same portions as those in FIG. 1A.

FIG. 4A illustrates an example in which a switching element including an oxide semiconductor (OS) is formed on a single crystal Si substrate in which a driver circuit of the display panel is formed and an organic EL element (OEL) electrically connected to the switching element is formed. In the case of a high-definition display, current flowing through a driving transistor of each pixel electrode is set to a level of nanoampere; accordingly, an OS transistor is suitable because current flows excessively in the case of using a transistor including silicon. In addition, for black display, off-state current is preferably low, and an OS transistor with low off-state current is suitable. In addition, in the case of using an organic EL element, a withstand voltage of 10 V or higher is desirably required, and an OS transistor is more suitable than the transistor including silicon. The emitted light 951 from the organic EL element (OEL) enables a user to recognize the displayed image. When a light-emitting element IR emitting infrared light and an OPD, or a light-receiving element, are formed in a layer including the organic EL element, infrared light is emitted and the light reflected by a fundus of a user's eye is received by the OPD, whereby image capturing of the fundus of the eye can be performed. When a light-receiving element is formed in the single crystalline Si substrate, infrared light is emitted and the light reflected by a fundus of a user's eye is received by a photodiode including silicon in a light-receiving region, whereby image capturing of the fundus of the eye may be performed.

FIG. 4B illustrates an example in which two single crystal Si substrates are bonded to each other. A first single crystal Si substrate in which a driver circuit or a control circuit is formed and a second single crystal Si substrate in which a switching element electrically connected to an organic EL element (OEL) is formed are bonded to each other with use of a known technique. When a light-receiving element is formed in the second single crystal Si substrate, infrared light is emitted and the light reflected by a fundus of a user's eye is received by a photodiode which includes silicon in a light-receiving region, whereby image capturing of the fundus of the eye may be performed.

For the outside area in FIG. 4B, an image captured by an external camera 91 provided in the electronic device 80 illustrated in FIG. 3C is displayed. In an AR type, positional adjustment for display is difficult; however, recognition of an image captured by the external camera 91 makes it possible to augment a reality. Furthermore, a battery 92 may be provided in a temple portion of glasses of the electronic device 80. The battery 92 has a function of supplying electric power to the external camera 91 or the display panel 280.

FIG. 4C illustrates an example in which a single crystal Si substrate is thinned. A switching element electrically connected to the light-receiving element and the organic EL element (OEL) are formed in a single crystal Si substrate in FIG. 4C, and a driver circuit or a control circuit are externally provided.

In the case where the single crystal Si substrate has a small thickness in each of FIG. 4A and FIG. 4C, visible light can be transmitted. Accordingly, an AR type electronic device which transmits external light can be provided.

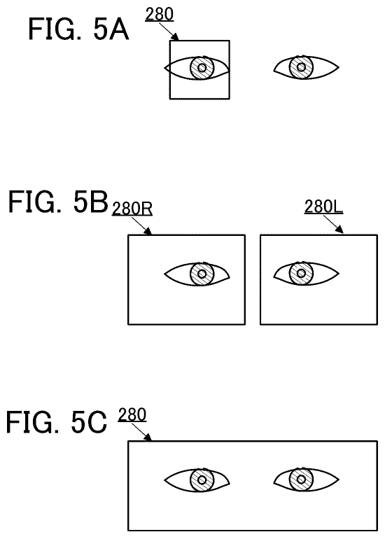

FIG. 5A, FIG. 5B, and FIG. 5C each illustrate a variation example of the size of the display panel 280. Note that although the shapes of edges of the display panel 280 are rectangular in FIG. 5A, FIG. 5B, and FIG. 5C, the shapes are not particularly limited thereto, and may be a circular shape or an elliptical shape.

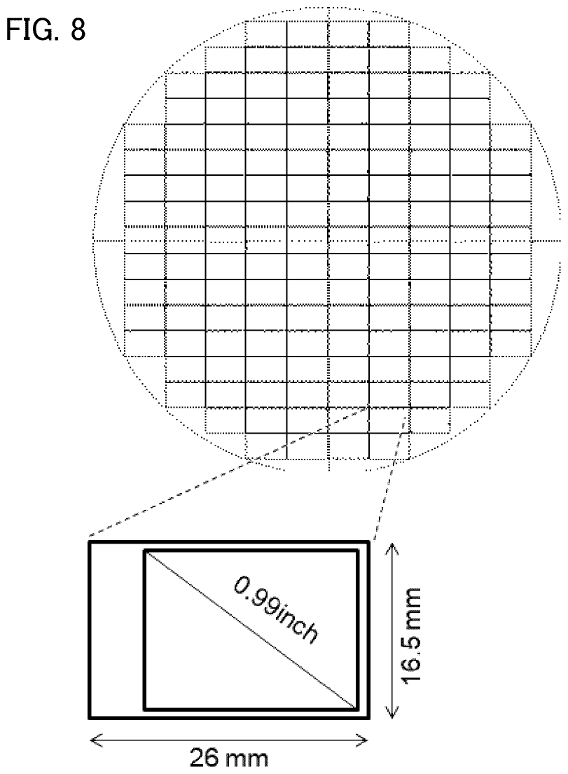

FIG. 5A illustrates an example in which the display panel overlaps with only one of the use's eyes when the display panel 280 is worn on a user's head, or an example of a single lens type. Note that in the case of capturing images of the fundi of both eyes, two electronic devices for the right eye and the left eye are prepared. In FIG. 5A, when a single crystal Si substrate is used in the structure of the display panel 280, the manufacturing cost can be reduced due to the small size. A plurality of chips can be extracted from one silicon wafer as illustrated in FIG. 8A. For example, in the case where a chip size is set to 26 mm×16.5 mm with use of a silicon wafer of 12 inches, 142 chips can be obtained from one wafer. A display panel with the screen size of 19.9 mm×14.9 mm (a diagonal size of 0.99 inches can be manufactured for one chip, and the number of pixels can be 1920×1440 and the resolution can be 2449 ppi. Furthermore, the electronic device itself can be light-weighted when the display panel 280 is worn on the user's head.

FIG. 5B illustrates an example of a type of glasses in which a display panel 280R for the right eye and a display panel 280L for the left eye are provided separately.

FIG. 5C illustrates an example in which one display panel 280 overlaps with both eyes and a screen size is larger than the other examples. The display panel 280 having a large area makes it possible to provide a large margin for position adjustment of the eyes and to be compatible with individual differences of users.

In the examples in FIG. 5A, FIG. 5B, and FIG. 5C, significant reductions in size and weight can be achieved as compared with those of a conventional ophthalmological diagnostic device.

An image displayed on the display screen is not always projected completely over a retina due to aberration of an eye. Brain builds the image by learning. In contrast, a retina pattern does not project a clear pattern over a sensor as in the conventional ophthalmological diagnostic device. In addition, authentication or health monitoring can be performed using a system utilizing AI.

The system utilizing AI is used in the following cases: a case in which arithmetic processing is performed inside an electronic device; a case in which captured image data is transmitted to the outside via a network and arithmetic processing is performed by an external arithmetic device; or a case combining those cases.

In the case of performing some or all of the arithmetic processing inside the electronic device, a control circuit needs to be provided in the single crystal Si substrate illustrated in FIG. 4A, FIG. 4B, and FIG. 4C. In the case of performing some of the arithmetic processing, an arithmetic circuit may be formed using a transistor including an oxide semiconductor (OS).

When data is transmitted to outside, a circuit for transmitting and receiving data is mounted on the electronic device, and the system utilizing AI is used.

Furthermore, a short-distance communication with a portable information terminal that a user has may be made possible. When an application using the system utilizing AI is downloaded to the portable information terminal, authentication or health monitoring can be performed by transmitting and receiving data with the portable information terminal.

As an example of the health monitoring, health monitoring of an eye can be performed using the display panel 280.

An image of a fundus of an eye is regularly captured when an electronic device including the display panel 280 is worn. At the time of taking the image of the fundus of the eye, a period when the display is darkened so that the user's eyes adapt to the dark is provided, and the time when the image cannot be taken during the period is the time of the eyes blinking or closing; accordingly, data on the number or timing of the blinks in the certain period is obtained and accumulated. Furthermore, when teacher data is created by the user inputting the degree of subjective symptoms of eye strain at the time of obtaining data, eye strain or abnormality of an eye can be detected on the basis of the accumulated data with use of the system utilizing AI.

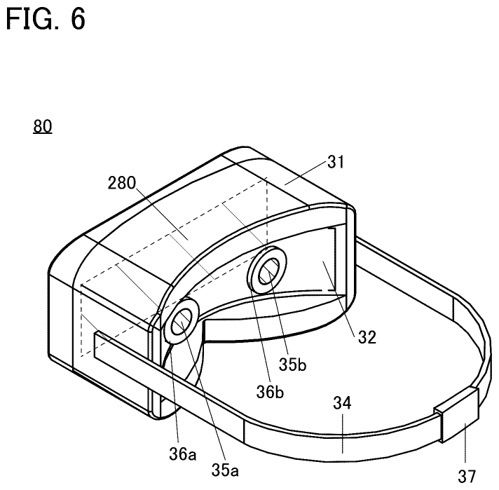

Although FIG. 3B illustrates an example of the glasses-type electronic device 80, there is no particular limitation as long as it is worn on the head; as illustrated in FIG. 6, the display panel 280 of the electronic device 80, which is referred to as a head-mounted display (HMD), may be mounted. Moreover, the electronic device 80 can also be called a goggle-type electronic device.

FIG. 6 illustrates an example in which the display panel 280 overlapping with both eyes as illustrated in FIG. 5C is used.

The electronic device 80 includes a housing 31, the display panel 280, a fixing unit 34, a battery 37, a pair of optical components (an optical component 35a and an optical component 35b), and a pair of frames (a frame 36a and a frame 36b). The battery 37 for driving the electronic device 80 supplies electric power to components of the electronic device 80 through a cable provided being incorporated in or in contact with the fixing unit 34. Although the battery 37 may be provided in the housing 31, it is preferably provided in the fixing unit 34, so that the center of gravity of the electronic device 80 is at the back, whereby the user can wear it more comfortably. Note that other than the battery 37, a driver circuit operating the display panel 280 may be provided in the fixing unit 34 to adjust the center of gravity of the electronic device 80.

The electronic device 80 includes an opening portion 32, and the optical components 35a and 35b and the frames 36a and 36b are provided to be in contact with the opening portion 32. The frames 36a and 36b are in contact with side surfaces of the optical components 35a and 35b, respectively and are provided to surround the optical components 35a and 35b, respectively. Moreover, the display panel 280 can be provided inside the housing 31.

The display panel 280 has a function of displaying an image. A user of the electronic device 80 can see an image displayed on the display panel 280 through the optical components 35a and 35b. The display panel 280 preferably has a function of displaying high-definition images. For example, when the display panel 280 has an 8-inch display region, the display device 280 preferably has a function of displaying images with a resolution of 8K4K.

The user of the electronic device 80 can see an image displayed on the display panel 280 through the optical components 35a and 35b. The electronic device 80 can be an electronic device capable of VR display.

The display panel 280 also has a function of detecting light. A light-receiving element which is a photoelectric conversion element is provided in the display panel 280.

When light emitted from the light-emitting element of the display panel 280 is delivered to a face of a user of the electronic device 80, for example, reflected light can be detected by the light-receiving element of the display panel 280. For example, the display panel 280 can have a function of detecting conditions of eyes and the periphery of the eyes of the user wearing the electronic device 80. Consequently, the electronic device 80 can have a function of recognizing the user's facial feature of the user's facial expression, and thus can have a function of estimating the degree of fatigue or emotion of the user, for instance. The electronic device 80 can detect, for example, watery eyes, blinks, or line of sight.

The display panel 280 preferably includes a light-emitting element, which is different from a light-emitting element for display, e.g., a light-emitting element emitting infrared light such as near-infrared light. In this case, the light-receiving element of the display panel 280 preferably has a function of detecting, e.g., infrared light such as near-infrared light. Accordingly, the electronic device 80 can capture an image of a fundus of a user's eye.

### Embodiment 2

In this embodiment, an example in which not an OPD but a light emitting diode (LED) is used as a light source 104 will be described.

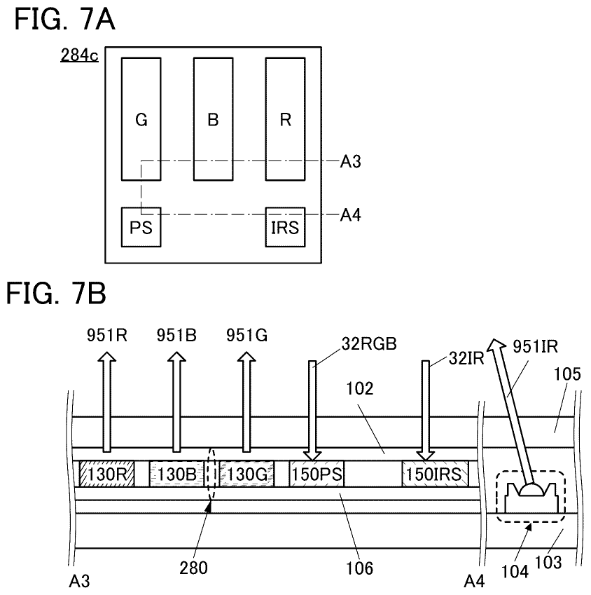

FIG. 7A illustrates an example of a pixel portion 284c which is different from the pixel portion 284a in FIG. 2B. In the example illustrated in FIG. 7A, the infrared light sensor IRS is provided in addition to the green (G) light-emitting element, the blue (B) light-emitting element, and the red (R) light-emitting element.

The display panel 280 in FIG. 7B corresponds to a cross-sectional structure taken along dashed-dotted line A3-A4 in FIG. 7A. The display panel 280 is included between the housing 103 and the protective member 105. Here, the display panel 280 illustrated in FIG. 7B has a function as a display device and a function of capturing an image of an object (a face, an iris, or a fundus of the eye of the user 81). In addition, a light emitting diode (LED) is used as the light source 104 in FIG. 7A and FIG. 7B. FIG. 7B illustrates an example in which the light source 104 is placed at a position not overlapping with the display panel 280. Here, the emitted light 951IR from the light source 104 is emitted outside the electronic device through the protective member 105. The display panel 280 includes a plurality of light-emitting devices and a plurality of light-receiving devices between a substrate 106 and a substrate 102. A subpixel (R) includes a light-emitting device 130R emitting the red light 951R. A subpixel (G) includes a light-emitting device 130G emitting the green light 951G. A subpixel (B) includes a light-emitting device 130B emitting the blue light 951B. Using these subpixels enables display of a full-color image with the display panel 280. In addition, a subpixel (PS) includes a light-receiving device 150PS and receives light 32RGB. Furthermore, the infrared light 951IR emitted from the light source 104 is reflected from an object (here, a fundus), and reflected light 32IR by the object is incident on a light-receiving device 150IRS. The object is not in contact with the electronic device; however, the object can be detected with use of the light-receiving device 150IRS.

In the case of using the display panel 280 illustrated in FIG. 7B, each of the light-emitting element 130R emitting the red light 951R, the light-emitting element 130G emitting the green light 951G, and the light-emitting element 130B emitting the blue light 951B are used as a light source to perform full-color display, and the light-emitting element emitting the emitted light 951IR, which is infrared light, is used as a light source to perform image capturing using infrared light.

When the optical system of the electronic device is made detachable, the electronic device can be used by switching between full-color display and fundus image capturing.

This embodiment can be freely combined with the other embodiments.

### Embodiment 3

In this embodiment, across-sectional structure examples of a semiconductor device 100A which constitutes the display panel 280 corresponding to FIG. 4A will be described.

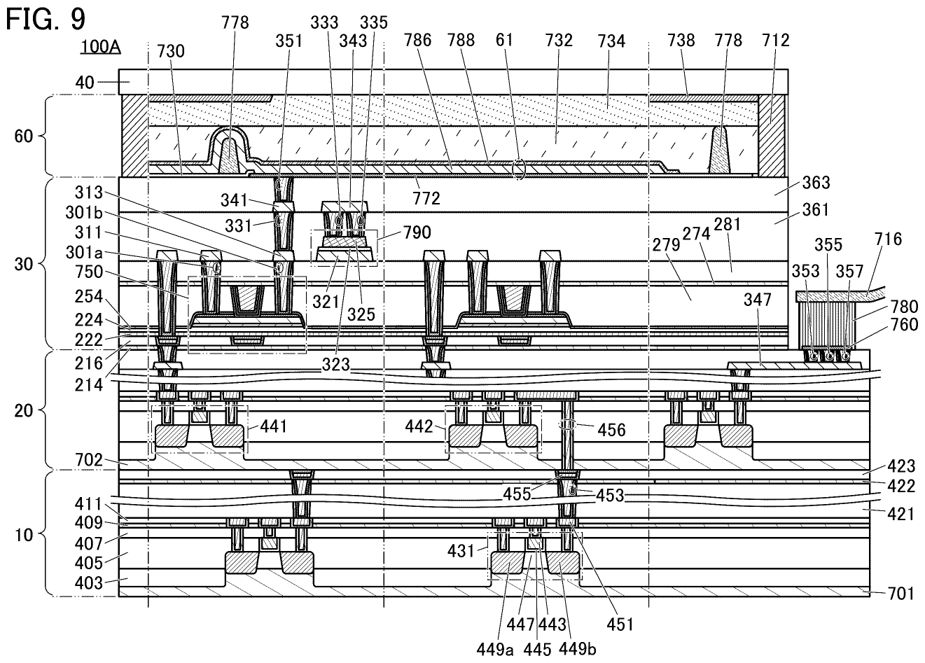

FIG. 9 is a cross-sectional view illustrating a structural example of the semiconductor device 100A and illustrates part of the semiconductor device 100A. As described above, the semiconductor device 100A is composed of a layer 10, a layer 20, a layer 30, a layer 60, and a sealing substrate 40.

### [Layer 10]

The layer 10 includes a substrate 701, and a transistor 431 is provided over the substrate 701. The transistor 431 is a transistor included in a memory cell, for example.

As the substrate 701, a single crystal semiconductor substrate typified by a single crystal silicon substrate can be used. Note that a semiconductor substrate other than a single crystal semiconductor substrate may be used as the substrate 701.

The transistor 431 includes a conductor 443 having a function as a gate electrode, an insulator 445 having a function as a gate insulator, and a part of the substrate 701. Part of the substrate 701 functions as a region including a channel formation region (a semiconductor region 447), a source region (one of a low-resistance region 449a and a low-resistance region 449b), and a drain region (the other of the low-resistance region 449a and the low-resistance region 449b) of the transistor 431. The transistor 431 may be either a p-channel transistor or an n-channel transistor.

When a single crystal semiconductor substrate typified by a single crystal silicon substrate is used as the substrate 701, the transistor 431 is a transistor that includes silicon in a channel formation region (such transistor is also referred to as “Si transistor”).

The transistor 431 is electrically isolated from other transistors by an element isolation layer 403. FIG. 9 illustrates the case where the transistor 431 and other transistors are electrically isolated from each other by the element isolation layer 403. The element isolation layer 403 can be formed by a LOCOS (LOCal Oxidation of Silicon) method or an STI (Shallow Trench Isolation) method.

Here, in the transistor 431, the semiconductor region 447 has a projecting shape. Moreover, the conductor 443 is provided to cover a side surface and a top surface of the semiconductor region 447 with the insulator 445 therebetween. Note that FIG. 9 does not illustrate the state where the conductor 443 covers the side surface of the semiconductor region 447. A material adjusting the work function can be used for the conductor 443.

A transistor having a projecting semiconductor region, like the transistor 431, can be referred to as a fin-type transistor because a projecting portion of a semiconductor substrate is used. Note that an insulator having a function of a mask for forming a projecting portion may be provided in contact with an upper portion of the projecting portion. Although FIG. 9 illustrates the structure in which the projecting portion is formed by processing part of the substrate 701, a semiconductor having a projecting shape may be formed by processing an SOI substrate.

Note that the structure of the transistor 431 illustrated in FIG. 9 is an example; the structure of the transistor 431 is not limited thereto and can be changed as appropriate in accordance with the circuit configuration or an operation method for the circuit. For example, the transistor 431 may be a planar transistor.

An insulator 405, an insulator 407, an insulator 409, and an insulator 411 are provided over the substrate 701, in addition to the element isolation layer 403 and the transistor 431. Furthermore, a conductor 451 is embedded in the insulator 405, the insulator 407, the insulator 409, and the insulator 411. Here, a top surface of the conductor 451 and a top surface of the insulator 411 can be substantially level with each other.

An insulator 421 and an insulator 422 are provided over the conductor 451 and the insulator 411. A conductor 453 is embedded in the insulator 421 and the insulator 422. Here, a top surface of the conductor 453 and a top surface of the insulator 422 can be substantially level with each other.

An insulator 423 is provided over the conductor 453 and the insulator 422. A conductor 455 is embedded in the insulator 423. Here, a top surface of the conductor 455 and a top surface of the insulator 423 can be substantially level with each other.

Note that insulators and conductors are stacked as necessary to make the layer 10 a multilayer wiring structure.

### [Layer 20]

The layer 20 includes a substrate 702, and a transistor 441 and a transistor 442 are provided over the substrate 702. The transistor 441 is, for example, a transistor included in a driver circuit for a display portion. The transistor 442 is, for example, a transistor included in a driver circuit for a memory portion.

As the substrate 702, a single crystal semiconductor substrate typified by a single crystal silicon substrate can be used like as the substrate 701. Note that a semiconductor substrate other than a single crystal semiconductor substrate may be used as the substrate 702. The layer 20 can have a structure similar to that of the layer 10. Thus, detailed description of the layer 20 is omitted.

In FIG. 9, the transistor 442 included in the layer 20 and the transistor 431 included in the layer 10 are electrically connected to each other through a conductor 456. The conductor 456 functions as a TSV. Note that the layer 10 and the layer 20 may be electrically connected to each other through a bump.

The layer 20 includes a conductor 760. The conductor 760 is a conductor included in a terminal portion. FIG. 9 illustrates an example in which the conductor 760 is electrically connected to an FPC 716 (FPC: Flexible Printed Circuit) through an anisotropic conductor 780. Various signals are supplied to the semiconductor device 100A through the FPC 716.

In addition, the conductor 760 is electrically connected to a conductor 347 included in the layer 20 through a conductor 353, a conductor 355, and a conductor 357. Although FIG. 9 illustrates three conductors, which are the conductor 353, the conductor 355, and the conductor 357, as conductors electrically connecting the conductor 760 and the conductor 347, one embodiment of the present invention is not limited thereto. The number of conductors electrically connecting the conductor 760 and the conductor 347 may be one, two, or four or more. Providing a plurality of conductors electrically connecting the conductor 760 and the conductor 347 can reduce the contact resistance.

### [Layer 30]

The layer 30 is provided over the layer 20. The layer 30 includes an insulator 214, and a transistor 750 is provided over the insulator 214. The transistor 750 is, for example, a transistor included in a pixel circuit. An OS transistor can be suitably used as the transistor 750. The OS transistor has a feature of extremely low off-state current. Accordingly, image data can be held for a longer time, so that the refresh operation can be less frequent. Thus, the power consumption of the semiconductor device 100A can be reduced.

A conductor 301a and a conductor 301b are embedded in an insulator 254, an insulator 279, an insulator 274, and an insulator 281. The conductor 301a is electrically connected to one of a source and a drain of the transistor 750, and the conductor 301b is electrically connected to the other of the source and the drain of the transistor 750. Here, top surfaces of the conductor 301a and the conductor 301b and a top surface of the insulator 281 can be substantially level with each other.

A conductor 311, a conductor 313, a conductor 331, a capacitor 790, a conductor 333, and a conductor 335 are embedded in the insulator 361. The conductor 311 and the conductor 313 are electrically connected to the transistor 750 and have a function of a wiring. The conductor 333 and the conductor 335 are electrically connected to the capacitor 790. Here, top surfaces of the conductor 331, the conductor 333, and the conductor 335 and a top surface of the insulator 361 can be substantially level with each other.

A conductor 341, a conductor 343, and a conductor 351 are embedded in the insulator 363. Here, a top surface of the conductor 351 and a top surface of the insulator 363 can be substantially level with each other.

Each of the insulator 405, the insulator 407, the insulator 409, the insulator 411, the insulator 421, the insulator 422, the insulator 423, the insulator 214, the insulator 279, the insulator 274, the insulator 281, the insulator 361, and the insulator 363 have a function of an interlayer film and may also have a function of a planarization film that covers unevenness thereunder. For example, a top surface of the insulator 363 may be planarized by planarization treatment using a chemical mechanical polishing (CMP) method to have the increased planarity.

As illustrated in FIG. 9, the capacitor 790 includes a lower electrode 321 and an upper electrode 325. An insulator 323 is provided between the lower electrode 321 and the upper electrode 325. In other words, the capacitor 790 has a stacked-layer structure in which the insulator 323 functioning as a dielectric is provided between the pair of electrodes. Note that although FIG. 9 illustrates an example in which the capacitor 790 is provided over the insulator 281, the capacitor 790 may be provided over an insulator different from the insulator 281.

In the example illustrated in FIG. 9, the conductor 301a and the conductor 301b are formed in the same layer. In the illustrated example, the conductor 311, the conductor 313, and the lower electrode 321 are formed in the same layer. In the illustrated example, the conductor 331, the conductor 333, and the conductor 335 are formed in the same layer. In the illustrated example, the conductor 341 and the conductor 343 are formed in the same layer. In the illustrated example, the conductor 353, the conductor 355, and the conductor 357 are formed in the same layer. Forming a plurality of conductors in the same layer simplifies the manufacturing process of the semiconductor device 100A and thus the manufacturing cost of the semiconductor device 100A can be reduced. Note that these conductors may be formed in different layers or may contain different types of materials.

### [Layer 60]

The layer 60 is provided over the layer 30. The layer 60 includes a light-emitting element 61. The light-emitting element 61 includes a conductor 772, an EL layer 786, and a conductor 788. The EL layer 786 contains an organic compound or an inorganic compound such as quantum dots.

Examples of materials that can be used as an organic compound include a fluorescent material and a phosphorescent material. Examples of materials that can be used as quantum dots include a colloidal quantum dot material, an alloyed quantum dot material, a core-shell quantum dot material, and a core quantum dot material.

The conductor 772 is electrically connected to the other of the source and the drain of the transistor 750 through the conductor 351, the conductor 341, the conductor 331, the conductor 313, and the conductor 301b. The conductor 772 is formed over the insulator 363 and has a function of a pixel electrode.

A material that transmits visible light or a material that reflects visible light can be used for the conductor 772. As a light-transmitting material, for example, an oxide material containing indium and zinc, an oxide material containing indium, gallium, and zinc (also referred to as “IGZO”), an oxide material containing indium, zinc, and tin (also referred to as “ITO”), and a material to which indium, zinc, tin, and silicon are added (also referred to as “ITSO”) can be used. Moreover, as a reflective material, for example, a material containing aluminum or silver can be used.

For example, in the case where light emitted by the light-emitting element 61 is emitted from the conductor 788 side, the conductor 772 preferably contains a reflective material. The conductor 772 may have a single-layer structure or a stacked-layer structure of a plurality of layers. For example, in the case where the conductor 772 is used as an anode, a three-layer structure in which silver is interposed between two layers of ITO may be employed.

In the case where silicon nitride is contained in a formation surface to be in contact with the conductor 772, a three-layer structure in which aluminum, titanium oxide, and ITO (or ITSO) are sequentially stacked from the formation surface side may be employed for the conductor 772. In the case where silicon nitride is contained in the formation surface to be in contact with the conductor 772, a two-layer structure in which aluminum and IGZO are sequentially stacked from the formation surface side may be employed for the conductor 772.

Note that the conductor 301a, the conductor 301b, the conductor 331, the conductor 351, the conductor 353, the conductor 355, the conductor 357, the conductor 453, the conductor 456, and the conductor 760 may have the structure similar to conductors 245a and 245b described in another embodiment. For example, the conductor 351 electrically connected to the light-emitting element 61 may be a conductor containing tungsten and titanium nitride. More specifically, a structure in which a side wall of the insulator 363 and tungsten are adjacent to each other with titanium nitride therebetween may be employed.

Although not illustrated in FIG. 9, an optical component (an optical substrate) such as a polarizing member, a retardation member, or an anti-reflection member can be provided in the semiconductor device 100A.

In the semiconductor device 100A illustrated in FIG. 9, an insulator 730 is provided over the insulator 363. Here, the insulator 730 can cover part of the conductor 772. Furthermore, using a light-transmitting material as the conductor 788 can make the light-emitting element 61 have a top-emission structure in which light is emitted to the conductor 788 side. Note that the light-emitting element 61 may have a bottom-emission structure in which light is emitted to the conductor 772 side or a dual-emission structure in which light is emitted towards both the conductor 772 and the conductor 788. Furthermore, a component 778 is provided between the insulator 730 and the EL layer 786.

[Sealing substrate 40]

The sealing substrate 40 is provided above the layer 30 to cover the display portion and the layer 60. The sealing substrate 40 is bonded to the layer 30 with a sealant 712. In the case where the light-emitting element 61 has a top-emission structure or a dual-emission structure, a material having a light-transmitting property is used for the sealing substrate 40.

Providing the sealing substrate 40 can prevent entry of impurities into the layer 60, and thus, the reliability of the semiconductor device 100A can be increased.

A light-blocking layer 738 is provided on the layer 60 side. The light-blocking layer 738 has a function of blocking light emitted from adjacent regions. Furthermore, the light-blocking layer 738 has a function of preventing external light from reaching the transistor 750. In FIG. 9, the light-blocking layer 738 is provided to have a region overlapping with the insulator 730.

The light-blocking layer 738 is covered with an insulator 734. The insulator 734 is provided as needed. In addition, although a solid sealing structure in which a filling layer 732 is provided between the light-emitting element 61 and the insulator 734 in this embodiment, a hollow sealing structure in which the filling layer 732 is not provided may be employed. In the case where the semiconductor device 100A has a hollow sealing structure, a part corresponding to the filling layer 732 may be filled with a Group 18 element (rare gas (noble gas)) and/or an inert gas containing nitrogen. In the case where light emitted from the light-emitting element 61 is emitted to the sealing substrate 40 side, a material having a light-transmitting property is preferably used for the filling layer 732.

### Modification Example 1

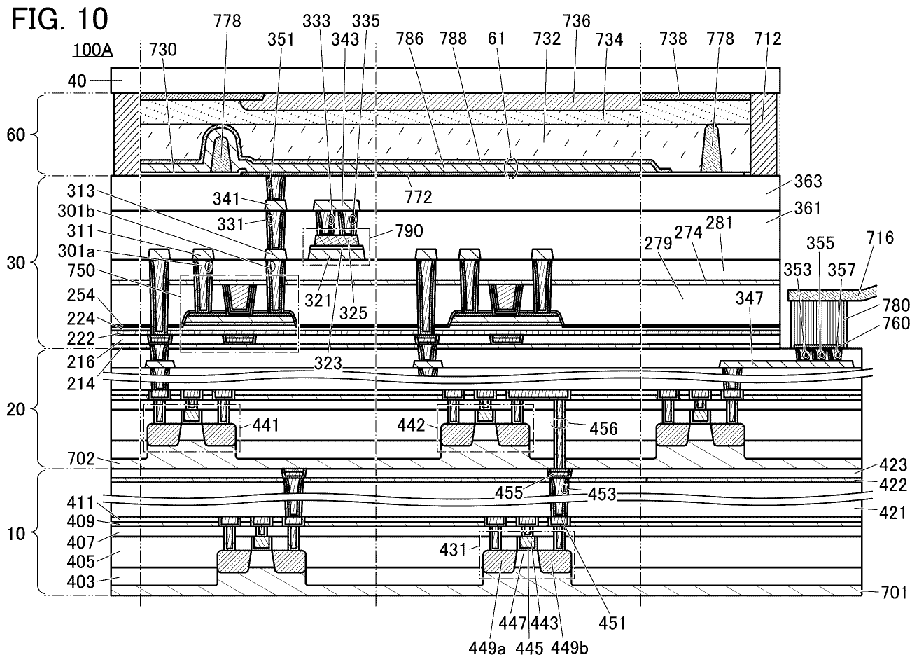

FIG. 10 illustrates a modification example of the semiconductor device 100A illustrated in FIG. 9. The semiconductor device 100A illustrated in FIG. 10 is different from the semiconductor device 100A illustrated in FIG. 9 in that a coloring layer 736 is provided. Note that the coloring layer 736 is provided to have a region overlapping with the light-emitting element 61. Providing the coloring layer 736 can improve the color purity of light extracted from the light-emitting element 61. Thus, the semiconductor device 100A can display high-quality images. Furthermore, all the light-emitting elements 61, for example, in the semiconductor device 100A can be light-emitting elements that emit white light; hence, the EL layers 786 are not necessarily formed by separate coloring, leading to higher definition of the semiconductor device 100A.

The light-emitting element 61 can have a micro optical resonator (microcavity) structure. Thus, light of predetermined colors (e.g., RGB) can be extracted without a coloring layer, and the semiconductor device 100A can perform color display. The structure without a coloring layer can suppress light absorption by the coloring layer. As a result, the semiconductor device 100A can display high-luminance images, and power consumption of the semiconductor device 100A can be reduced. Note that a structure in which a coloring layer is not provided can be employed even when the EL layer 786 is formed into an island shape for each pixel or into a stripe shape for each pixel column, i.e., the EL layers 786 are formed separately for each color. Note that the luminance of the semiconductor device 100A can be, for example, higher than or equal to 500 cd/m² and lower than or equal to 20000 cd/m², preferably higher than or equal to 1000 cd/m² and lower than or equal to 20000 cd/m², further preferably higher than or equal to 5000 cd/m² and lower than or equal to 20000 cd/m².

### Modification Example 2

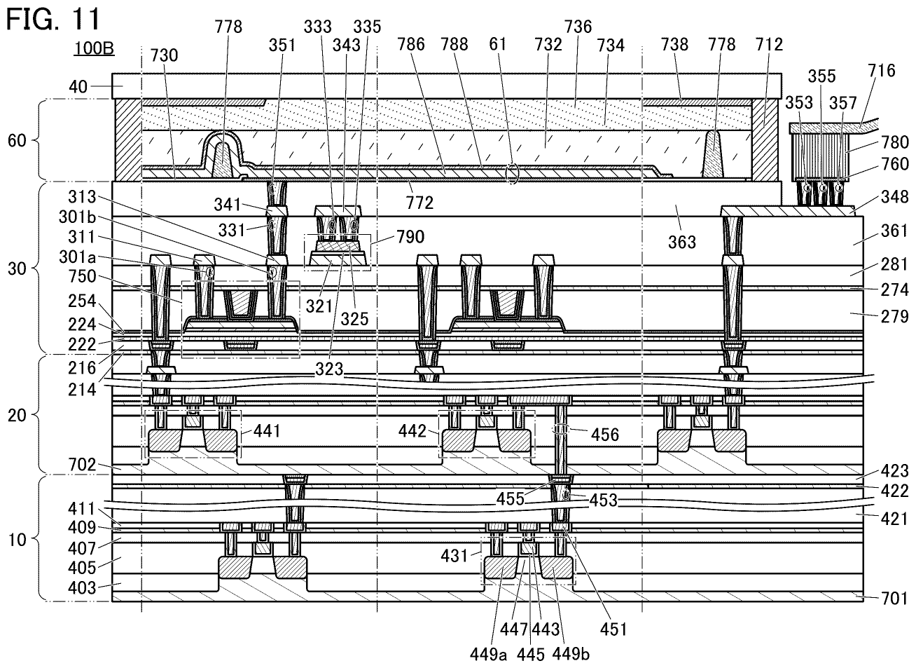

FIG. 11 illustrates a cross-sectional structure example of a semiconductor device 100B, which is a modification example of the semiconductor device 100A. In the cross-sectional structure example of the semiconductor device 100B illustrated in FIG. 11, a conductor 348 is provided over the insulator 361 included in the layer 30 instead of the conductor 347.

The conductor 348 is electrically connected to the conductor 760 through the conductor 353, the conductor 355, and the conductor 357. The conductor 348 functions like the conductor 347.

### Modification Example 3

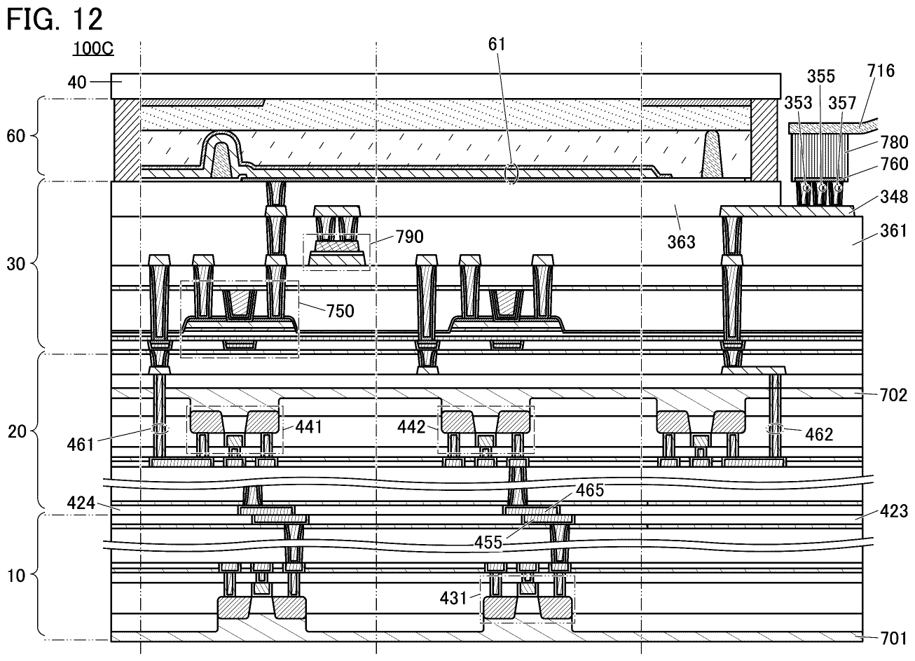

FIG. 12 illustrates a cross-sectional structure example of a structure where the layer 30 is stacked over the layer 10 and overlaps with the layer 10 with the layer 20 therebetween. FIG. 12 illustrates a cross-sectional structure example of a semiconductor device 100C, which is a modification example of the semiconductor device 100B. In FIG. 12, the layer 20 is provided over the layer 10 to overlap with each other so that a transistor included in the layer 20 and a transistor included in the layer 10 face each other. Accordingly, the layer 30 is provided on the substrate 702 side in the layer 20.

The conductor included in the 10 layer and a conductor included in the layer 20 can be electrically connected to each other by Cu—Cu bonding, for example. In FIG. 12, for example, the conductor 455 included in the layer 10 and a conductor 465 included in the layer 20 are electrically connected to each other by Cu—Cu coupling. In this case, each of the conductor 455 and the conductor 465 is formed using a conductor containing Cu (copper). Furthermore, the insulator 423 which the conductor 455 is provided to be embedded in and an insulator 424 which the conductor 465 is provided to be embedded in are preferably insulators containing the same element. For example, each of the insulator 423 and the insulator 424 may be silicon oxide or silicon oxynitride. When the insulator 423 and the insulator 424 are insulators containing the same element, the attachment strength of the layer 10 and the layer 20 are increased. Before performing attachment of the layer 10 and the layer 20, surfaces of both layers to be attached are preferably subjected to CMP treatment to increase planarity of the both surfaces.

Moreover, in FIG. 12, a conductor included in the layer 10 and a conductor included in the layer 20 may be electrically connected to each other through a TSV. For example, a conductor 461 and a conductor 462, which are included in the layer 20, each are a TSV that penetrates the substrate 702.

### Modification Example 4

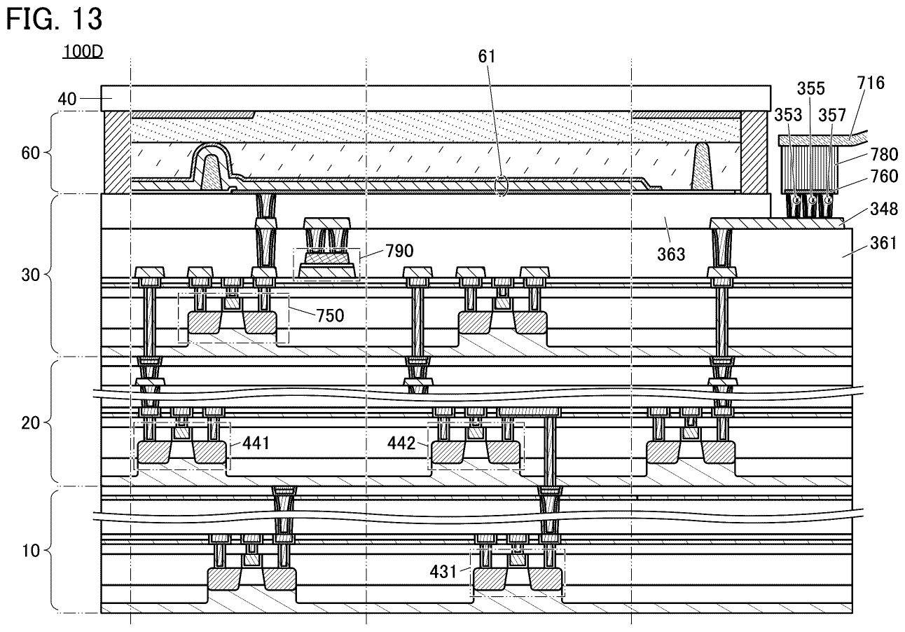

FIG. 13 illustrates a cross-sectional structure example of a semiconductor device 100D, which is a modification example of the semiconductor device 100C. FIG. 13 is a semiconductor device corresponding to the display panel 280 in FIG. 4C. The cross-sectional structure example in FIG. 13 illustrates an example in which transistors included in the layer 30 are Si transistors.

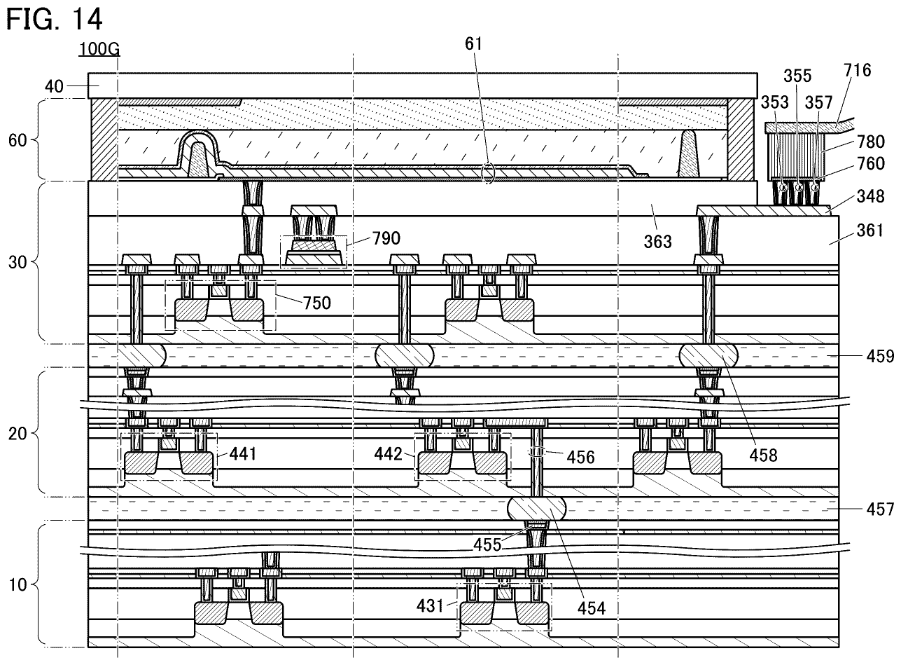

As illustrated in FIG. 14, a bump 454 and an adhesive layer 457 may be provided between the layer 10 and the layer 20. FIG. 14 illustrates a cross-sectional structure example of a semiconductor device 100G, which is a modification example of the semiconductor device 100D. FIG. 14 is a semiconductor device corresponding to the display panel 280 in FIG. 4B. The layer 10 and the layer 20 are fixed to each other with the adhesive layer 457 and are electrically connected to each other through the bump 454. In FIG. 14, the conductor 456 and the conductor 455 are electrically connected to each other through the bump 454. Similarly, a bump 458 and an adhesive layer 459 may be provided between the layer 20 and the layer 30. The layer 20 and the layer 30 are fixed to each other with the adhesive layer 459 and are electrically connected to each other through the bump 458. Note that the number of the bump 454 that electrically connects the layer 10 and the layer 20 is not limited to one and may be two or more. The number of the bump 458 that electrically connects the layer 20 and the layer 30 is not limited to one and may be two or more.

Also in each of the semiconductor device 100A, the semiconductor device 100B, the semiconductor device 100C, the semiconductor device 100D, and the semiconductor device 100G, transistors other than the OS transistors (e.g., Si transistors) may be used for the transistors included in the layer 30. As transistors included in the layer 10, the layer 20, and the layer 30, various transistors can be used depending on the purpose or the usage.

The structures described in this embodiment can be used in an appropriate combination with the structures described in the other embodiments.

### Embodiment 4

In this embodiment, a light-emitting element 61 (also referred to as “light-emitting device”) will be described.

### Structure Example of Light-Emitting Element

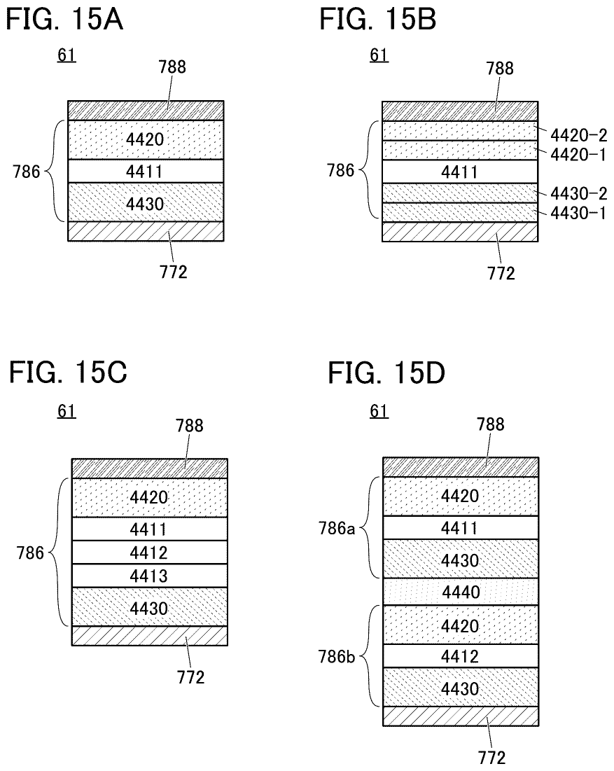

As illustrated in FIG. 15A, the light-emitting element 61 includes an EL layer 786 between a pair of electrodes (a conductor 772 and a conductor 788). The EL layer 786 can be formed of a plurality of layers such as a layer 4420, a light-emitting layer 4411, or a layer 4430. The layer 4420 can include, for example, a layer containing a substance with a high electron-injection property (an electron-injection layer) and a layer containing a substance with a high electron-transport property (an electron-transport layer). The light-emitting layer 4411 contains a light-emitting compound, for example. The layer 4430 can include, for example, a layer containing a substance with a high hole-injection property (a hole-injection layer) and a layer containing a substance with a high hole-transport property (a hole-transport layer).

The structure including the layer 4420, the light-emitting layer 4411, and the layer 4430, which is provided between a pair of electrodes, can function as a single light-emitting unit, and the structure in FIG. 15A is referred to as a single structure in this specification.

FIG. 15B shows a modification example of the EL layer 786 included in the light-emitting element 61 illustrated in FIG. 15A. Specifically, the light-emitting element 61 illustrated in FIG. 15B includes a layer 4430-1 over the conductor 772, a layer 4430-2 over the layer 4430-1, the light-emitting layer 4411 over the layer 4430-2, a layer 4420-1 over the light-emitting layer 4411, a layer 4420-2 over the layer 4420-1, and the conductor 788 over the layer 4420-2. For example, when the conductor 772 functions as an anode and the conductor 788 functions as a cathode, the layer 4430-1 functions as a hole-injection layer, the layer 4430-2 functions as a hole-transport layer, the layer 4420-1 functions as an electron-transport layer, and the layer 4420-2 functions as an electron-injection layer. Alternatively, when the conductor 772 functions as a cathode and the conductor 788 functions as an anode, the layer 4430-1 functions as an electron-injection layer, the layer 4430-2 functions as an electron-transport layer, the layer 4420-1 functions as a hole-transport layer, and the layer 4420-2 functions as a hole-injection layer. With such a layer structure, carriers can be efficiently injected to the light-emitting layer 4411, and the efficiency of the recombination of carriers in the light-emitting layer 4411 can be enhanced.

Note that the structure in which a plurality of light-emitting layers (the light-emitting layer 4411, a light-emitting layer 4412, and a light-emitting layer 4413) are provided between the layer 4420 and the layer 4430 as illustrated in FIG. 15C is also an example of the single structure.

The structure in which a plurality of light-emitting units (an EL layer 786a and an EL layer 786b) are connected in series with an intermediate layer (charge-generation layer) 4440 therebetween as illustrated in FIG. 15D is referred to as a tandem structure or a stack structure in this specification. The tandem structure enables a light-emitting element capable of high luminance light emission.

In the case where the light-emitting element 61 has the tandem structure illustrated in FIG. 15D, the EL layer 786a and the EL layer 786b may emit light of the same color. For example, the EL layer 786a and the EL layer 786b may both emit green light. Note that in the case where the display portion includes three subpixels of R, G, and B and each of the subpixels includes a light-emitting element, the tandem structure may be employed for the light-emitting element of each subpixels. Specifically, the EL layer 786a and the EL layer 786b in the subpixel of R each contain a material capable of emitting red light, the EL layer 786a and the EL layer 786b in the subpixel of G each contain a material capable of emitting green light, and the EL layer 786a and the EL layer 786b in the subpixel of B each contain a material capable of emitting blue light. In other words, the light-emitting layer 4411 and the light-emitting layer 4412 may contain the same material. When the EL layer 786a and the EL layer 786b emit light of the same color, the current density per unit emission luminance can be reduced. Thus, the reliability of the light-emitting element 61 can be increased.

The emission color of the light-emitting element can be red, green, blue, cyan, magenta, yellow, or white depending on the material that constitutes the EL layer 786. Furthermore, the color purity can be further increased when the light-emitting element has a microcavity structure.

The light-emitting layer may contain two or more light-emitting substances that emit light of R (red), G (green), B (blue), Y (yellow), or O (orange). The light-emitting element that emits white light (also referred to as “white light-emitting device”) preferably contains two or more kinds of light-emitting substances in the light-emitting layer. To obtain white light emission, two or more kinds of light-emitting substances are selected such that their emission colors are complementary. For example, when the emission color of a first light-emitting layer and the emission color of a second light-emitting layer have a relationship of complementary colors, it is possible to obtain the light-emitting element which emits white light as a whole. This can be applied to a light-emitting element including three or more light-emitting layers.

The light-emitting layer preferably contains two or more light-emitting substances that emit light of R (red), G (green), B (blue), Y (yellow), and O (orange). Alternatively, the light-emitting layer preferably contains two or more light-emitting substances that emit light containing two or more of spectral components of R, G, and B.

### Formation Method of Light-Emitting Element 61

A method for forming the light-emitting element 61 will be described below.

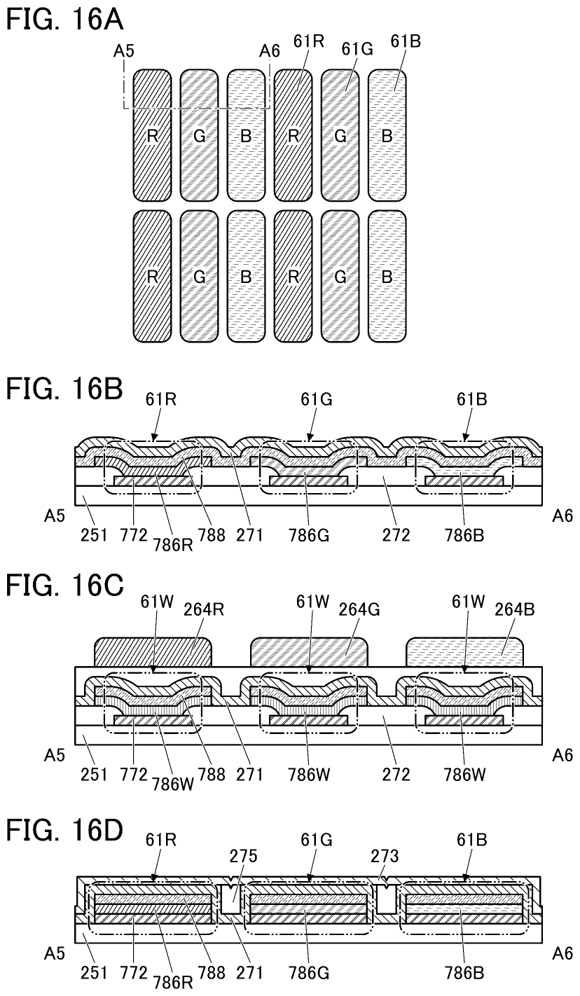

FIG. 16A illustrates a schematic top view of the light-emitting element 61. The light-emitting element 61 includes a plurality of light-emitting elements 61R exhibiting red, a plurality of light-emitting elements 61G exhibiting green, and a plurality of light-emitting elements 61B exhibiting blue. In FIG. 16A, light-emitting regions of the light-emitting elements are denoted by R, G, and B to easily differentiate the light-emitting elements. Note that the structure of the light-emitting element 61 illustrated in FIG. 16A may be referred to as an SBS (Side By Side) structure. Although the structure illustrated in FIG. 16A has three colors of red (R), green (G), and blue (B), one embodiment of the present invention is not limited thereto. For example, the structure may have four or more colors.

The light-emitting elements 61R, the light-emitting elements 61G, and the light-emitting elements 61B are arranged in a matrix. FIG. 16A illustrates what is called a stripe arrangement, in which the light-emitting elements of the same color are arranged in one direction. Note that the arrangement method of the light-emitting elements is not limited thereto; another arrangement method such as a delta arrangement, a zigzag arrangement, or a PenTile arrangement may also be used.

As the light-emitting element 61R, the light-emitting element 61G, and the light-emitting element 61B, an organic EL device typified by an OLED (Organic Light Emitting Diode) or a QLED (Quantum-dot Light Emitting Diode) is preferably used. As a light-emitting substance contained in the EL element, a substance that emits fluorescence (a fluorescent material), a substance that emits phosphorescence (a phosphorescent material), an inorganic compound (a quantum dot material), and a substance that exhibits thermally activated delayed fluorescence (a thermally activated delayed fluorescent (TADF) material) can be given.

FIG. 16B is a schematic cross-sectional view taken along dashed-dotted line A5-A6 in FIG. 16A. FIG. 16B illustrates cross sections of the light-emitting element 61R, the light-emitting element 61G, and the light-emitting element 61B. The light-emitting element 61R, the light-emitting element 61G, and the light-emitting element 61B are each provided over an insulating layer 251 and include the conductor 772 functioning as a pixel electrode and the conductor 788 functioning as a common electrode. For the insulating layer 251, one or both of an inorganic insulating film and an organic insulating film can be used. An inorganic insulating film is preferably used as the insulating layer 251. As the inorganic insulating film, for example, an oxide insulating film and a nitride insulating film such as a silicon oxide film, a silicon oxynitride film, a silicon nitride oxide film, a silicon nitride film, an aluminum oxide film, an aluminum oxynitride film, or a hafnium oxide film can be given.

The light-emitting element 61R includes an EL layer 786R between the conductor 772 functioning as a pixel electrode and the conductor 788 functioning as a common electrode. The EL layer 786R contains at least a light-emitting organic compound that emits light with an intensity in a red wavelength range. An EL layer 786G included in the light-emitting element 61G contains at least a light-emitting organic compound that emits light with an intensity in a green wavelength range. An EL layer 786B included in the light-emitting element 61B contains at least a light-emitting organic compound that emits light with an intensity in a blue wavelength range.

The EL layer 786R, the EL layer 786G, and the EL layer 786B may each include one or more of an electron-injection layer, an electron-transport layer, a hole-injection layer, and a hole-transport layer in addition to the layer containing a light-emitting organic compound (the light-emitting layer).

The conductor 772 functioning as a pixel electrode is provided in each of the light-emitting elements. The conductor 788 functioning as a common electrode is provided as a continuous layer shared by the light-emitting elements. A conductive film that transmits visible light is used for either the conductor 772 functioning as a pixel electrode or the conductor 788 functioning as a common electrode, and a reflective conductive film is used for the other. When the conductor 772 functioning as a pixel electrode has a light-transmitting property and the conductor 788 functioning as a common electrode has a reflective property, a bottom-emission display device can be obtained, whereas when the conductor 772 functioning as a pixel electrode has a reflective property and the conductor 788 functioning as a common electrode has a light-transmitting property, a top-emission display device can be obtained. Note that when both the conductor 772 functioning as a pixel electrode and the conductor 788 functioning as a common electrode have a light-transmitting property, a dual-emission display device can be obtained.

An insulating layer 272 is provided to cover end portions of the conductor 772 functioning as a pixel electrode. End portions of the insulating layer 272 are preferably tapered. For the insulating layer 272, a material similar to the material that can be used for the insulating layer 251 can be used.

The EL layer 786R, the EL layer 786G, and the EL layer 786B each include a region in contact with a top surface of the conductor 772 functioning as a pixel electrode and a region in contact with a surface of the insulating layer 272. End portions of the EL layer 786R, the EL layer 786G, and the EL layer 786B are positioned over the insulating layer 272.

As illustrated in FIG. 16B, there is a gap between the two EL layers of the light-emitting elements of different colors. In this manner, the EL layer 786R, the EL layer 786G, and the EL layer 786B are preferably provided so as not to be in contact with each other. This suitably prevents unintentional light emission (also referred to as crosstalk) from being caused by a current flowing through two adjacent EL layers. As a result, the contrast can be increased to achieve a display device with high display quality.

The EL layer 786R, the EL layer 786G, and the EL layer 786B can be formed separately by a vacuum evaporation method using a shadow mask typified by a metal mask. Alternatively, these layers may be formed separately by a photolithography method. The use of the photolithography method achieves a display device with high resolution, which is difficult to obtain in the case of using a metal mask.

A protective layer 271 is provided over the conductor 788 functioning as a common electrode so as to cover the light-emitting element 61R, the light-emitting element 61G, and the light-emitting element 61B. The protective layer 271 has a function of preventing diffusion of impurities typified by water into the light-emitting elements from above.

The protective layer 271 can have, for example, a single-layer structure or a stacked-layer structure at least including an inorganic insulating film. As the inorganic insulating film, for example, an oxide film or a nitride film, typified by a silicon oxide film, a silicon oxynitride film, a silicon nitride oxide film, a silicon nitride film, an aluminum oxide film, an aluminum oxynitride film, or a hafnium oxide film can be given. Alternatively, a semiconductor material typified by indium gallium oxide or indium gallium zinc oxide (IGZO) may be used for the protective layer 271. Note that the protective layer 271 may be formed by an ALD method, a CVD method, or a sputtering method. Although the protective layer 271 includes an inorganic insulating film in this example, one embodiment of the present invention is not limited thereto. For example, the protective layer 271 may have a stacked-layer structure of an inorganic insulating film and an organic insulating film.

Note that in this specification, a nitride oxide refers to a compound that contains more nitrogen than oxygen. An oxynitride refers to a compound that contains more oxygen than nitrogen. The content of each element can be measured by Rutherford backscattering spectrometry (RBS), for example.

In the case where an indium gallium zinc oxide is used for the protective layer 271, the indium gallium zinc oxide can be processed by a wet etching method or a dry etching method. For example, in the case where IGZO is used as the protective layer 271, a chemical solution typified by oxalic acid, phosphoric acid, or a mixed chemical solution (e.g., a mixed chemical solution of phosphoric acid, acetic acid, nitric acid, and water, which is also referred to as a mixed acid aluminum etchant) can be used. Note that the volume ratio of phosphoric acid, acetic acid, nitric acid, and water mixed in the mixed acid aluminum etchant can be 53.3:6.7:3.3:36.7 or in the neighborhood thereof.

FIG. 16C illustrates an example different from the above. Specifically, in FIG. 16C, light-emitting elements 61W that emit white light are provided. The light-emitting elements 61W each include an EL layer 786W that emits white light between the conductor 772 functioning as a pixel electrode and the conductor 788 functioning as a common electrode.

The EL layer 786W can have, for example, a structure in which two or more light-emitting layers that are selected so as to emit light of complementary colors are stacked. It is also possible to use a stacked EL layer in which a charge-generation layer is provided between light-emitting layers.

FIG. 16C illustrates three light-emitting elements 61W side by side. A coloring layer 264R is provided above the light-emitting element 61W on the left. The coloring layer 264R functions as a band path filter that transmits red light. Similarly, a coloring layer 264G that transmits green light is provided above the light-emitting element 61W in the middle, and a coloring layer 264B that transmits blue light is provided above the light-emitting element 61W on the right. Thus, the display device can display an image with colors.

Here, the EL layer 786W and the conductor 788 functioning as a common electrode are each separated between two adjacent light-emitting elements 61W. This can prevent unintentional light emission from being caused by a current flowing through the EL layers 786W of the two adjacent light-emitting elements 61W. Particularly when stacked EL layers in which a charge-generation layer is provided between two light-emitting layers are used as the EL layer 786W, crosstalk is more significant as the resolution increases, i.e., as the distance between adjacent pixels decreases, leading to lower contrast. Thus, the above structure can achieve a display device having both high resolution and high contrast.

The EL layer 786W and the conductor 788 functioning as a common electrode are preferably isolated by a photolithography method. This can reduce the distance between light-emitting elements, achieving a display device with a higher aperture ratio than that formed using, for example, a shadow mask such as a metal mask.

Note that in the case of a bottom-emission light-emitting element, a coloring layer may be provided between the conductor 772 functioning as a pixel electrode and the insulating layer 251.

FIG. 16D illustrates an example different from the above. Specifically, in FIG. 16D, the insulating layers 272 are not provided between the light-emitting element 61R, the light-emitting element 61G, and the light-emitting element 61B. With such a structure, the display device can have a high aperture ratio. The protective layer 271 covers side surfaces of the EL layer 786R, the EL layer 786G, and the EL layer 786B. With this structure, impurities (typically, water) can be inhibited from entering the EL layer 786R, the EL layer 786G, and the EL layer 786B through their side surfaces. In the structure illustrated in FIG. 16D, the top shapes of the conductor 772, the EL layer 786R, and the conductor 788 are substantially aligned with each other. This structure can be formed in such a manner that the conductor 772, the EL layer 786R, and the conductor 788 are formed and collectively processed using a resist mask. In this process, the EL layer 786R and the conductor 788 are processed using the conductor 788 as a mask, and thus this process can be called self-alignment patterning. Although the EL layer 786R is described here, the EL layer 786G and the EL layer 786B can each have a similar structure.

In FIG. 16D, a protective layer 273 is further provided over the protective layer 271. For example, the protective layer 271 can be formed with an apparatus that can deposit a film with excellent coverage (typically, an ALD apparatus), and the protective layer 273 can be formed with an apparatus that can deposit a film with coverage inferior to that of the protective layer 271 (typically, a sputtering apparatus), whereby a space 275 can be provided between the protective layer 271 and the protective layer 273. In other words, the spaces 275 are positioned between the EL layer 786R and the EL layer 786G and between the EL layer 786G and the EL layer 786B.

Note that the space 275 includes, for example, any one or more selected from air, nitrogen, oxygen, carbon dioxide, and Group 18 elements (typically, helium, neon, argon, xenon, and krypton). Furthermore, for example, a gas used during the deposition of the protective layer 273 is sometimes included in the space 275. For example, in the case where the protective layer 273 is deposited by a sputtering method, any one or more of the above-described Group 18 elements is sometimes included in the space 275. In the case where a gas is included in the space 275, a gas can be identified with a gas chromatography method. Alternatively, in the case where the protective layer 273 is deposited by a sputtering method, a gas used in the sputtering is sometimes contained in the protective layer 273. In this case, an element such as argon is sometimes detected when the protective layer 273 is analyzed by an energy dispersive X-ray analysis (EDX analysis).

In the case where the refractive index of the space 275 is lower than that of the protective layer 271, light emitted from the EL layer 786R, the EL layer 786G, or the EL layer 786B is reflected at the interface between the protective layer 271 and the space 275. Thus, light emitted from the EL layer 786R, the EL layer 786G, or the EL layer 786B can be inhibited from entering an adjacent pixel in some cases. This can inhibit color mixture of light emitted from adjacent pixels and thus can improve the display quality of the display device.

In the case of the structure illustrated in FIG. 16D, a region between the light-emitting element 61R and the light-emitting element 61G or a region between the light-emitting element 61G and the light-emitting element 61B (hereinafter simply referred to as a distance between the light-emitting elements) can be small. Specifically, the distance between the light-emitting elements can be less than or equal to 1 μm, preferably less than or equal to 500 nm, further preferably less than or equal to 200 nm, less than or equal to 100 nm, less than or equal to 90 nm, less than or equal to 70 nm, less than or equal to 50 nm, less than or equal to 30 nm, less than or equal to 20 nm, less than or equal to 15 nm, or less than or equal to 10 nm. In other words, the display device includes a region in which an interval between the side surface of the EL layer 786R and the side surface of the EL layer 786G or an interval between the side surface of the EL layer 786G and the side surface of the EL layer 786B is less than or equal to 1 μm, preferably less than or equal to 0.5 μm (500 nm), further preferably less than or equal to 100 nm.

In the case where the space 275 includes a gas, for example, the structure illustrated in FIG. 16D can be referred to as an air isolation structure. The air isolation structure allows the light-emitting elements to be isolated from each other and color mixing of light or crosstalk between the light-emitting elements can be inhibited.

Alternatively, the space 275 may be filled with a filler. Examples of the filler include an epoxy resin, an acrylic resin, a silicone resin, a phenol resin, a polyimide resin, an imide resin, a PVC (polyvinyl chloride) resin, a PVB (polyvinyl butyral) resin, and an EVA (ethylene vinyl acetate) resin. Alternatively, a photoresist may be used as the filler. The photoresist used as the filler may be either a positive photoresist or a negative photoresist.

In the case where the space 275 is filled with a filler, an inorganic insulating material and an organic insulating material suitably combined with each other. Specifically, a stacked-layer structure in which aluminum oxide and a photoresist over the aluminum oxide is provided can be given as an example. Note that the above-described aluminum oxide is suitably formed by an ALD method, leading to an increase in coverage.

When the white-light-emitting device (having a single structure or a tandem structure) and a light-emitting device having an SBS structure are compared to each other, the light-emitting device having an SBS structure can have lower power consumption than the white-light-emitting device. To reduce power consumption, a light-emitting device having an SBS structure is preferably used. Meanwhile, the white-light-emitting device is suitable in terms of lower manufacturing cost or higher manufacturing yield because the manufacturing process of the white-light-emitting device is simpler than that of the light-emitting device having an SBS structure.

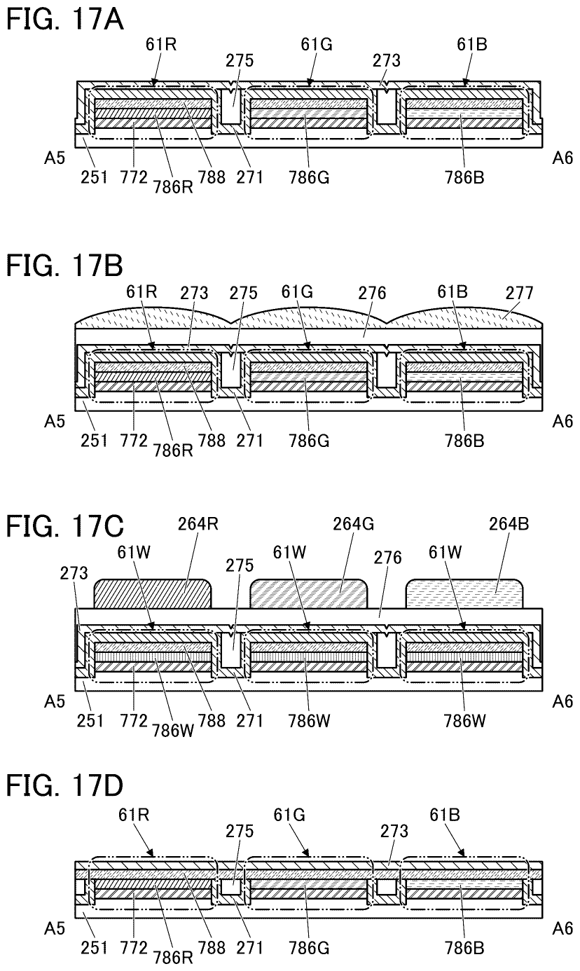

FIG. 17A illustrates an example different from the above. Specifically, the structure illustrated in FIG. 17A is different from the structure illustrated in FIG. 16D in the structure of the insulating layer 251. The insulating layer 251 has a recessed portion in its top surface that is formed by being partially etched when the light-emitting element 61R, the light-emitting element 61G, and the light-emitting element 61B are processed. In addition, the protective layer 271 is formed in the recessed portion. In other words, in the cross-sectional view, a region is provided, in which the bottom surface of the protective layer 271 is positioned below the bottom surface of the conductor 772. With the region, impurities (typically, water) can be suitably inhibited from entering the light-emitting element 61R, the light-emitting element 61G, and the light-emitting element 61B from the bottom. It is likely that the recessed portion can be formed when impurities (also referred to as residue) that could be attached to the side surfaces of the light-emitting element 61R, the light-emitting element 61G, and the light-emitting element 61B in processing of the light-emitting elements are removed by wet etching. After the residue is removed, the side surfaces of the light-emitting elements are covered with the protective layer 271, whereby a highly reliable display device can be provided.

FIG. 17B illustrates an example different from the above. Specifically, the structure illustrated in FIG. 17B includes an insulating layer 276 and a microlens array 277 in addition to the structure illustrated in FIG. 17A. The insulating layer 276 functions as an adhesive layer. Note that when the refractive index of the insulating layer 276 is lower than that of the microlens array 277, the microlens array 277 can condense light emitted from the light-emitting element 61R, the light-emitting element 61G, and the light-emitting element 61B. This can increase the light extraction efficiency of the display device. In particular, this is suitable, because a user can see bright images when the user sees the display surface from the front of the display device. As the insulating layer 276, a variety of curable adhesives, e.g., a photocurable adhesive such as an ultraviolet curable adhesive, a reactive curable adhesive, a thermosetting adhesive, and an anaerobic adhesive can be used. Examples of these adhesives include an epoxy resin, an acrylic resin, a silicone resin, a phenol resin, a polyimide resin, an imide resin, a PVC (polyvinyl chloride) resin, a PVB (polyvinyl butyral) resin, and an EVA (ethylene vinyl acetate) resin. In particular, a material with low moisture permeability, such as an epoxy resin, is preferred. Alternatively, a two-component resin may be used. An adhesive sheet may be used.

FIG. 17C illustrates an example different from the above. Specifically, the structure illustrated in FIG. 17C includes three light-emitting elements 61W instead of the light-emitting element 61R, the light-emitting element 61G, and the light-emitting element 61B in the structure illustrated in FIG. 17A. In addition, the insulating layer 276 is provided over the three light-emitting elements 61W, and the coloring layer 264R, the coloring layer 264G, and the coloring layer 264B are provided over the insulating layer 276. Specifically, the coloring layer 264R that transmits red light is provided at a position overlapping with the light-emitting element 61W on the left, the coloring layer 264G that transmits green light is provided at a position overlapping with the light-emitting element 61W in the middle, and the coloring layer 264B that transmits blue light is provided at a position overlapping with the light-emitting element 61W on the right. Thus, the semiconductor device can display an image with colors. The structure illustrated in FIG. 17C is also a modification example of the structure illustrated in FIG. 16C.

FIG. 17D illustrates an example different from the above. Specifically, in the structure illustrated in FIG. 17D, the protective layer 271 is provided to be adjacent to the side surfaces of the conductor 772 and the EL layer 786. The conductor 788 is provided as a continuous layer shared by the light-emitting elements. In the structure illustrated in FIG. 17D, the space 275 is preferably filled with a filler.

Furthermore, the color purity of emitted light can be further increased when the light-emitting element 61 has a microcavity structure. In order that the light-emitting element 61 has a microcavity structure, a product of a distance d between the conductor 772 and the conductor 788 and a refractive index n of the EL layer 786 (optical path length) is set to m times half of a wavelength λ (m is an integer of 1 or more). The distance d can be obtained by Formula 1.

d=m×λ/(2×n) Formula 1

According to Formula 1, in the light-emitting element 61 having the microcavity structure, the distance d is determined in accordance with the wavelength (emission color) of emitted light. The distance d corresponds to the thickness of the EL layer 786. Thus, the EL layer 786G is provided to have a larger thickness than the EL layer 786B, and the EL layer 786R is provided to have a larger thickness than the EL layer 786G in some cases.

To be exact, the distance d is a distance from a reflection region in the conductor 772 functioning as a reflective electrode to a reflection region in the conductor 788 functioning as a transflective electrode. For example, in the case where the conductor 772 is a stack of silver and ITO that is a transparent conductive film and the ITO is positioned on the EL layer 786 side, the distance d suitable for the emission color can be set by adjusting the thickness of the ITO. That is, even when the EL layer 786R, the EL layer 786G, and the EL layer 786B have the same thickness, the distance d suitable for the emission color can be obtained by adjusting the thickness of the ITO.

However, it is sometimes difficult to determine the exact position of the reflection region in each of the conductor 772 and the conductor 788. In this case, it is assumed that the effect of the microcavity structure can be fully obtained with a certain position in each of the conductor 772 and the conductor 788 being supposed as the reflection region.

The light-emitting element 61 includes a hole-transport layer, a hole-transport layer, a light-emitting layer, an electron-transport layer, and an electron-injection layer. Note that a specific structure example of the light-emitting element 61 will be described in another embodiment. In order to increase the outcoupling efficiency in the microcavity structure, the optical path length from the conductor 772 functioning as a reflective electrode to the light-emitting layer is preferably set to an odd multiple of λ/4. In order to achieve this optical distance, the thicknesses of the layers in the light-emitting element 61 are preferably adjusted as appropriate.

In the case where light is emitted from the conductor 788 side, the reflectance of the conductor 788 is preferably higher than the transmittance thereof. The light transmittance of the conductor 788 is preferably higher than or equal to 2% and lower than or equal to 50%, further preferably higher than or equal to 2% and lower than or equal to 30%, still further preferably higher than or equal to 2% and lower than or equal to 10%. When the transmittance of the conductor 788 is set low (the reflectance is set high), the effect of the microcavity structure can be enhanced.

The structures described in this embodiment can be used in an appropriate combination with the structures described in the other embodiments.

### Embodiment 5

In this embodiment, transistors that can be used in the semiconductor device of one embodiment of the present invention will be described.

### Structure Example of Transistor

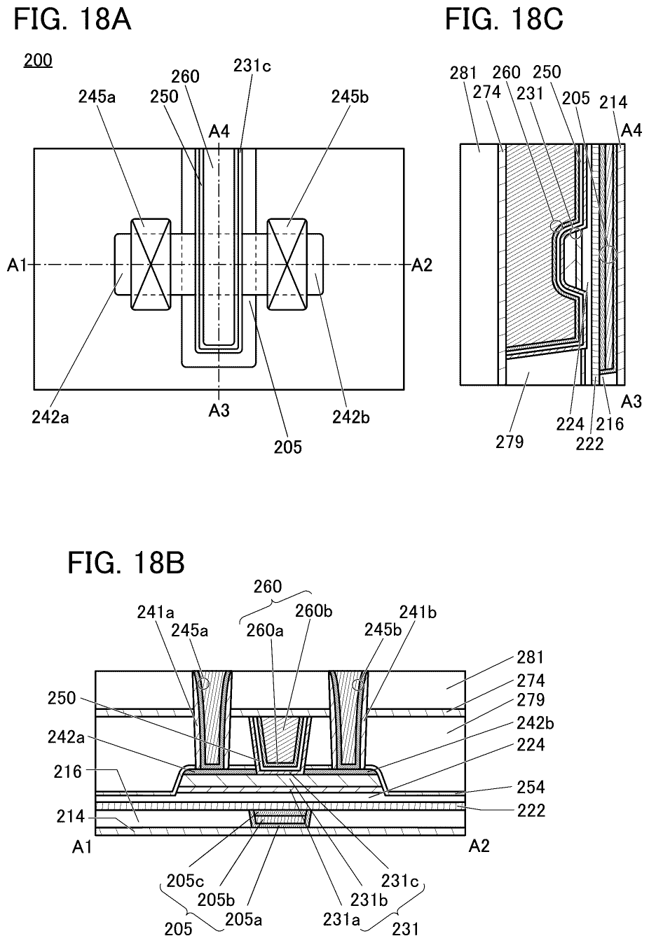

FIG. 18A, FIG. 18B, and FIG. 18C are a top view and cross-sectional views of a transistor 200 that can be used in the display device of one embodiment of the present invention and the periphery of the transistor 200. The transistor 200 can be used in the display device of one embodiment of the present invention. For example, the transistor 200 can be used as the transistor included in the layer 30.

FIG. 18A is a top view of the transistor 200. FIG. 18B and FIG. 18C are cross-sectional views of the transistor 200. Here, FIG. 18B is a cross-sectional view of a portion indicated by dashed-dotted line A1-A2 in FIG. 18A and is a cross-sectional view of the transistor 200 in the channel length direction. FIG. 18C is a cross-sectional view of a portion indicated by dashed-dotted line A3-A4 in FIG. 18A and is a cross-sectional view of the transistor 200 in the channel width direction. Note that some components are omitted in the top view of FIG. 18A for clarity of the drawing.

As illustrated in FIG. 18, the transistor 200 includes a metal oxide 231a placed over a substrate (not illustrated); a metal oxide 231b placed over the metal oxide 231a; a conductor 242a and a conductor 242b that are placed apart from each other over the metal oxide 231b; the insulator 279 that is placed over the conductor 242a and the conductor 242b and has an opening between the conductor 242a and the conductor 242b; a conductor 260 placed in the opening; an insulator 250 placed between the conductor 260 and each of the metal oxide 231b, the conductor 242a, the conductor 242b, and the insulator 279; and a metal oxide 231c placed between the insulator 250 and each of the metal oxide 231b, the conductor 242a, the conductor 242b, and the insulator 279. Here, as illustrated in FIG. 18B and FIG. 18C, preferably, a top surface of the conductor 260 is substantially aligned with top surfaces of the insulator 250, the insulator 254, the metal oxide 231c, and the insulator 279. Hereinafter, the metal oxide 231a, the metal oxide 231b, and the metal oxide 231c may be collectively referred to as a metal oxide 231.

In the transistor 200 illustrated in FIG. 18, side surfaces of the conductor 242a and the conductor 242b on the conductor 260 side are substantially perpendicular. Note that the transistor 200 illustrated in FIG. 18 is not limited thereto, and the angle formed between the side surfaces and bottom surfaces of the conductor 242a and the conductor 242b may be greater than or equal to 10° and less than or equal to 80°, preferably greater than or equal to 30° and less than or equal to 60°. The side surfaces of the conductor 242a and the conductor 242b that face each other may have a plurality of surfaces.

As illustrated in FIG. 18, the insulator 254 is preferably placed between the insulator 279 and each of an insulator 224, the metal oxide 231a, the metal oxide 231b, the conductor 242a, the conductor 242b, and the metal oxide 231c. Here, as illustrated in FIG. 18B and FIG. 18C, the insulator 254 is preferably in contact with a side surface of the metal oxide 231c, a top surface and the side surface of the conductor 242a, a top surface and the side surface of the conductor 242b, side surfaces of the metal oxide 231a and the metal oxide 231b, and a top surface of the insulator 224.

In the transistor 200, three layers of the metal oxide 231a, the metal oxide 231b, and the metal oxide 231c are stacked in and around a region where the channel is formed (hereinafter also referred to as channel formation region); however, the present invention is not limited thereto. For example, a two-layer structure of the metal oxide 231b and the metal oxide 231c or a stacked-layer structure of four or more layers may be employed. Although the conductor 260 is illustrated to have a stacked-layer structure of two layers in the transistor 200, the present invention is not limited thereto. For example, the conductor 260 may have a single-layer structure or a stacked-layer structure of three or more layers. Furthermore, each of the metal oxide 231a, the metal oxide 231b, and the metal oxide 231c may have a stacked-layer structure of two or more layers.

For example, in the case where the metal oxide 231c has a stacked-layer structure including a first metal oxide and a second metal oxide over the first metal oxide, the first metal oxide preferably has a composition similar to that of the metal oxide 231b and the second metal oxide preferably has a composition similar to that of the metal oxide 231a.

Here, the conductor 260 functions as a gate electrode of the transistor, and the conductor 242a and the conductor 242b each function as a source electrode or a drain electrode. As described above, the conductor 260 is formed to be embedded in the opening of the insulator 279 and the region interposed between the conductor 242a and the conductor 242b. Here, the positions of the conductor 260, the conductor 242a, and the conductor 242b are selected in a self-aligned manner with respect to the opening of the insulator 279. In other words, in the transistor 200, the gate electrode can be placed between the source electrode and the drain electrode in a self-aligned manner. Thus, the conductor 260 can be formed without an alignment margin, resulting in a reduction in the area occupied by the transistor 200. Accordingly, the display device can have higher resolution. In addition, the display device can have a narrow bezel.

As illustrated in FIG. 18, the conductor 260 preferably includes a conductor 260a provided on the inner side of the insulator 250 and a conductor 260b provided to be embedded on the inner side of the conductor 260a.

The transistor 200 preferably includes the insulator 214 placed over the substrate (not illustrated); an insulator 216 placed over the insulator 214; a conductor 205 placed to be embedded in the insulator 216; an insulator 222 placed over the insulator 216 and the conductor 205; and the insulator 224 placed over the insulator 222. The metal oxide 231a is preferably placed over the insulator 224.

The insulator 274 and the insulator 281 functioning as interlayer films are preferably placed over the transistor 200. Here, the insulator 274 is preferably placed in contact with the top surfaces of the conductor 260, the insulator 250, the insulator 254, the metal oxide 231c, and the insulator 279.

The insulator 222, the insulator 254, and the insulator 274 preferably have a function of inhibiting diffusion of at least one of hydrogen (e.g., a hydrogen atom and a hydrogen molecule). For example, the insulator 222, the insulator 254, and the insulator 274 preferably have a lower hydrogen permeability than the insulator 224, the insulator 250, and the insulator 279. Moreover, the insulator 222 and the insulator 254 preferably have a function of inhibiting diffusion of at least one of oxygen (e.g., an oxygen atom and an oxygen molecule). For example, the insulator 222 and the insulator 254 preferably have a lower oxygen permeability than the insulator 224, the insulator 250, and the insulator 279.

Here, the insulator 224, the metal oxide 231, and the insulator 250 are separated from the insulator 279 and the insulator 281 by the insulator 254 and the insulator 274. This can inhibit entry of impurities such as hydrogen contained in the insulator 279 and the insulator 281 and excess oxygen into the insulator 224, the metal oxide 231, and the insulator 250.

A conductor 245a and a conductor 245b each of which is electrically connected to the transistor 200 and functions as a plug is preferably provided. Note that insulators (an insulator 241a and an insulator 241b) are provided in contact with side surfaces of the conductor 245a and 245b functioning as plugs. In other words, the insulator 241a and 241b are provided in contact with the inner walls of openings in the insulator 254, the insulator 279, the insulator 274, and the insulator 281. In addition, a structure may be employed in which a first conductor of each of the conductor 245a and 245b is provided in contact with side surfaces of the insulator 241a and 241b and a second conductor of each of the conductor 245a and 245b is provided on the inner side of the first conductor. Here, top surfaces of the conductor 245a and 245b and a top surface of the insulator 281 can be substantially level with each other. Although the transistor 200 has a structure in which the first conductor of the conductor 245a and 245b and the second conductor of the conductor 245a and 245b are stacked, the present invention is not limited thereto. For example, each of the conductor 245a and 245b may have a single-layer structure or a stacked-layer structure of three or more layers. In the case where a component has a stacked-layer structure, layers may be distinguished by ordinal numbers corresponding to the formation order.

In the transistor 200, a metal oxide functioning as an oxide semiconductor (hereinafter also referred to as an oxide semiconductor) is preferably used as the metal oxide 231 including the channel formation region (the metal oxide 231a, the metal oxide 231b, and the metal oxide 231c). For example, it is preferable to use a metal oxide having a band gap of 2 eV or more, preferably 2.5 eV or more as the metal oxide to be the channel formation region of the metal oxide 231.

The metal oxide preferably contains at least indium (In) or zinc (Zn). In particular, indium (In) and zinc (Zn) are preferably contained. In addition to them, an element M is preferably contained. As the element M, one or more of aluminum (Al), gallium (Ga), yttrium (Y), tin (Sn), boron (B), titanium (Ti), iron (Fe), nickel (Ni), germanium (Ge), zirconium (Zr), molybdenum (Mo), lanthanum (La), cerium (Ce), neodymium (Nd), hafnium (Hf), tantalum (Ta), tungsten (W), magnesium (Mg), and cobalt (Co) can be used. In particular, the element M is preferably one or more of aluminum (Al), gallium (Ga), yttrium (Y), and tin (Sn). Furthermore, the element M preferably contains one or both of gallium (Ga) and tin (Sn).

As illustrated in FIG. 18B, the metal oxide 231b in a region that does not overlap with the conductor 242a and the conductor 242b sometimes has a smaller thickness than the metal oxide 231b in a region that overlaps with the conductor 242a and the conductor 242b. The thin region is formed when part of the top surface of the metal oxide 231b is removed at the time of forming the conductor 242a and the conductor 242b. When a conductive film to be the conductor 242a and the conductor 242b is formed, a low-resistance region is sometimes formed on a top surface of the metal oxide 231b in the vicinity of the interface with the conductive film. Removing the low-resistance region positioned between the conductor 242a and the conductor 242b on the top surface of the metal oxide 231b in the above manner can prevent formation of the channel in the region.

According to one embodiment of the present invention, a display device that includes small-size transistors and has high resolution can be provided. A display device that includes a transistor with a high on-state current and has high luminance can be provided. A display device that includes a transistor operating at high speed and thus operates at high speed can be provided. A display device that includes a transistor having stable electrical characteristics and is highly reliable can be provided. A display device that includes a transistor with a low off-state current and has low power consumption can be provided.

The structure of the transistor 200 that can be used in the display device of one embodiment of the present invention is described in detail.

The conductor 205 is placed to include a region overlapping with the metal oxide 231 and the conductor 260. Furthermore, the conductor 205 is preferably provided to be embedded in the insulator 216.

The conductor 205 includes a conductor 205a, a conductor 205b, and a conductor 205c. The conductor 205a is provided in contact with the bottom surface and a side wall of the opening provided in the insulator 216. The conductor 205b is provided to be embedded in a recessed portion formed by the conductor 205a. Here, the top surface of the conductor 205b is lower in level than the top surface of the conductor 205a and the top surface of the insulator 216. The conductor 205c is provided in contact with the top surface of the conductor 205b and the side surface of the conductor 205a. Here, the top surface of the conductor 205c is substantially level with the top surface of the conductor 205a and the top surface of the insulator 216. That is, the conductor 205b is surrounded by the conductor 205a and the conductor 205c.

For the conductor 205a and the conductor 205c, it is preferable to use a conductive material having a function of inhibiting diffusion of impurities such as a hydrogen atom, a hydrogen molecule, a water molecule, a nitrogen atom, a nitrogen molecule, a nitrogen oxide molecule (N₂O, NO, or NO₂), and a copper atom. Alternatively, it is preferable to use a conductive material having a function of inhibiting diffusion of oxygen (e.g., at least one of an oxygen atom and an oxygen molecule).

When the conductor 205a and the conductor 205c are formed using a conductive material having a function of inhibiting diffusion of hydrogen, impurities typified by hydrogen contained in the conductor 205b can be inhibited from diffusing into the metal oxide 231 through the insulator 224. When the conductor 205a and the conductor 205c are formed using a conductive material having a function of inhibiting diffusion of oxygen, the conductivity of the conductor 205b can be inhibited from being lowered because of oxidation. As the conductive material having a function of inhibiting diffusion of oxygen, for example, titanium, titanium nitride, tantalum, tantalum nitride, ruthenium, or ruthenium oxide is preferably used. Thus, the conductor 205a is a single layer or stacked layers of the above conductive materials. For example, titanium nitride is used for the conductor 205a.

For the conductor 205b, a conductive material containing tungsten, copper, or aluminum as its main component is preferably used. For example, tungsten is used for the conductor 205b.

The conductor 260 sometimes functions as a first gate (also referred to as top gate) electrode. The conductor 205 sometimes functions as a second gate (also referred to as bottom gate) electrode. In that case, by changing a potential applied to the conductor 205 not in synchronization with but independently of a potential applied to the conductor 260, Vth of the transistor 200 can be controlled. In particular, by applying a negative potential to the conductor 205, Vth of the transistor 200 can be higher than 0 V and the off-state current can be made low. Thus, a drain current at the time when a potential applied to the conductor 260 is 0 V can be lower in the case where a negative potential is applied to the conductor 205 than in the case where the negative potential is not applied to the conductor 205.

The conductor 205 is preferably provided to be larger than the channel formation region in the metal oxide 231. In particular, it is preferable that the conductor 205 extend to the outside beyond an end portion of the metal oxide 231 that intersects with the channel width direction, as illustrated in FIG. 18C. In other words, the conductor 205 and the conductor 260 preferably overlap with each other with the insulator placed therebetween, in a region outside the side surface of the metal oxide 231 in the channel width direction.

With the above structure, the channel formation region of the metal oxide 231 can be electrically surrounded by electric fields of the conductor 260 having a function of a first gate electrode and electric fields of the conductor 205 having a function of a second gate electrode.

Furthermore, as illustrated in FIG. 18C, the conductor 205 extends to function as a wiring as well. However, without limitation to this structure, a structure in which a conductor functioning as a wiring is provided below the conductor 205 may be employed.

The insulator 214 preferably functions as a barrier insulating film that inhibits the entry of impurities typified by water or hydrogen to the transistor 200 from the substrate side. Accordingly, it is preferable to use, for the insulator 214, an insulating material having a function of inhibiting diffusion of impurities typified by a hydrogen atom, a hydrogen molecule, a water molecule, a nitrogen atom, a nitrogen molecule, a nitrogen oxide molecule (N₂O, NO, or NO₂), and a copper atom (an insulating material through which the impurities are less likely to pass). Alternatively, it is preferable to use an insulating material having a function of inhibiting diffusion of at least one of oxygen (e.g., oxygen atoms and oxygen molecules) (an insulating material through which the oxygen is less likely to pass).

For example, aluminum oxide or silicon nitride is preferably used for the insulator 214. Accordingly, it is possible to inhibit diffusion of impurities typified by water or hydrogen to the transistor 200 side from the substrate side of the insulator 214. Alternatively, it is possible to inhibit diffusion of oxygen contained in the insulator 224 to the substrate side of the insulator 214.

The permittivity of each of the insulator 216, the insulator 279, and the insulator 281 functioning as an interlayer film is preferably lower than that of the insulator 214. When a material with a low permittivity is used for an interlayer film, the parasitic capacitance generated between wirings can be reduced. For the insulator 216, the insulator 279, and the insulator 281, for example, silicon oxide, silicon oxynitride, silicon nitride oxide, silicon nitride, silicon oxide to which fluorine is added, silicon oxide to which carbon is added, silicon oxide to which carbon and nitrogen are added, or porous silicon oxide can be used as appropriate.

The insulator 222 and the insulator 224 each have a function of a gate insulator.

Here, the insulator 224 in contact with the metal oxide 231 preferably releases oxygen by heating. In this specification, oxygen that is released by heating is referred to as excess oxygen in some cases. For example, silicon oxide or silicon oxynitride can be used as appropriate for the insulator 224. When an insulator containing oxygen is provided in contact with the metal oxide 231, oxygen vacancies in the metal oxide 231 can be reduced, leading to improved reliability of the transistor 200.

Specifically, an oxide material that releases part of oxygen by heating is preferably used for the insulator 224. An oxide that releases oxygen by heating is an oxide film in which the amount of released oxygen converted into oxygen atoms is greater than or equal to 1.0×10¹⁸ atoms/cm³, preferably greater than or equal to 1.0×10¹⁹ atoms/cm³, further preferably greater than or equal to 2.0×10¹⁹ atoms/cm³ or greater than or equal to 3.0×10²⁰ atoms/cm³ in TDS (Thermal Desorption Spectroscopy) analysis. Note that the temperature of the film surface in the TDS analysis is preferably in the range of 100° C. to 700° C., inclusive or 100° C. to 400° C., inclusive.

As illustrated in FIG. 18C, the insulator 224 is sometimes thinner in a region that overlaps with neither the insulator 254 nor the metal oxide 231b than in the other regions. In the insulator 224, the region that overlaps with neither the insulator 254 nor the metal oxide 231b preferably has a thickness with which the above oxygen can be adequately diffused.

Like the insulator 214, the insulator 222 preferably functions as a barrier insulating film that inhibits the entry of impurities typified by water or hydrogen into the transistor 200 from the substrate side. For example, the insulator 222 preferably has a lower hydrogen permeability than the insulator 224. When the insulator 224, the metal oxide 231, and the insulator 250 are surrounded by the insulator 222, the insulator 254, and the insulator 274, the entry of impurities typified by water or hydrogen into the transistor 200 from outside can be inhibited.

Furthermore, it is preferable that the insulator 222 have a function of inhibiting diffusion of at least one of oxygen (e.g., an oxygen atom and an oxygen molecule) (it is preferable that the oxygen be less likely to pass through the insulator 222). For example, the insulator 222 preferably has a lower oxygen permeability than the insulator 224. The insulator 222 preferably has a function of inhibiting diffusion of oxygen and impurities, in which case oxygen contained in the metal oxide 231 is less likely to diffuse to the substrate side. Moreover, the conductor 205 can be inhibited from reacting with oxygen contained in the insulator 224 and the metal oxide 231.

As the insulator 222, an insulator containing an oxide of one or both of aluminum and hafnium, which is an insulating material, is preferably used. As the insulator containing an oxide of one or both of aluminum and hafnium, aluminum oxide, hafnium oxide, or an oxide containing aluminum and hafnium (hafnium aluminate) is preferably used. In the case where the insulator 222 is formed using such a material, the insulator 222 functions as a layer inhibiting release of oxygen from the metal oxide 231 and entry of impurities typified by hydrogen into the metal oxide 231 from the periphery of the transistor 200.

Alternatively, aluminum oxide, bismuth oxide, germanium oxide, niobium oxide, silicon oxide, titanium oxide, tungsten oxide, yttrium oxide, or zirconium oxide may be added to these insulators, for example. Alternatively, these insulators may be subjected to nitriding treatment. Silicon oxide, silicon oxynitride, or silicon nitride may be stacked over the above insulator.

The insulator 222 may be a single layer or a stacked layer using an insulator containing what is called a high-k material, such as aluminum oxide, hafnium oxide, tantalum oxide, zirconium oxide, lead zirconate titanate (PZT), strontium titanate (SrTiO₃), or (Ba,Sr)TiO₃ (BST). With further miniaturization and higher integration of a transistor, a problem such as generation of leakage current may arise because of a thinned gate insulator. When a high-k material is used for the insulator functioning as a gate insulator, a gate potential at the time of operation of the transistor can be reduced while the physical thickness is maintained.

Note that the insulator 222 and the insulator 224 may each have a stacked-layer structure of two or more layers. In that case, without limitation to a stacked-layer structure formed of the same material, a stacked-layer structure formed of different materials may be employed. For example, an insulator similar to the insulator 224 may be provided below the insulator 222.

The metal oxide 231 includes the metal oxide 231a, the metal oxide 231b over the metal oxide 231a, and the metal oxide 231c over the metal oxide 231b. When the metal oxide 231 includes the metal oxide 231a under the metal oxide 231b, it is possible to inhibit diffusion of impurities into the metal oxide 231b from the components formed below the metal oxide 231a. Moreover, when the metal oxide 231 includes the metal oxide 231c over the metal oxide 231b, it is possible to inhibit diffusion of impurities into the metal oxide 231b from the components formed above the metal oxide 231c.

Note that the metal oxide 231 preferably has a stacked-layer structure of a plurality of oxide layers that differ in the atomic ratio of metal atoms. For example, in the case where the metal oxide 231 contains at least indium (In) and the element M, the proportion of the number of atoms of the element M contained in the metal oxide 231a to the number of atoms of all elements that constitute the metal oxide 231a is preferably higher than the proportion of the number of atoms of the element M contained in the metal oxide 231b to the number of atoms of all elements that constitute the metal oxide 231b. In addition, the atomic ratio of the element M to In in the metal oxide 231a is preferably greater than the atomic ratio of the element M to In in the metal oxide 231b. Here, a metal oxide that can be used as the metal oxide 231a or the metal oxide 231b can be used as the metal oxide 231c.

The energy of the conduction band minimum of each of the metal oxide 231a and the metal oxide 231c is preferably higher than the energy of the conduction band minimum of the metal oxide 231b. In other words, the electron affinity of each of the metal oxide 231a and the metal oxide 231c is preferably smaller than the electron affinity of the metal oxide 231b. In this case, a metal oxide that can be used as the metal oxide 231a is preferably used as the metal oxide 231c. Specifically, the proportion of the number of atoms of the element M contained in the metal oxide 231c to the number of atoms of all elements that constitute the metal oxide 231c is preferably higher than the proportion of the number of atoms of the element M contained in the metal oxide 231b to the number of atoms of all elements that constitute the metal oxide 231b. In addition, the atomic ratio of the element M to In in the metal oxide 231c is preferably greater than the atomic ratio of the element M to In in the metal oxide 231b.

Here, the energy level of the conduction band minimum gently changes at junction portions between the metal oxide 231a, the metal oxide 231b, and the metal oxide 231c. In other words, at junction portions between the metal oxide 231a, the metal oxide 231b, and the metal oxide 231c, the energy level of the conduction band minimum continuously changes or the energy levels are continuously connected. This can be achieved by decreasing the density of defect states in a mixed layer formed at the interface between the metal oxide 231a and the metal oxide 231b and the interface between the metal oxide 231b and the metal oxide 231c.

Specifically, when the metal oxide 231a and the metal oxide 231b or the metal oxide 231b and the metal oxide 231c contain the same element (as a main component) in addition to oxygen, a mixed layer with a low density of defect states can be formed. For example, an In—Ga—Zn oxide, a Ga—Zn oxide, or gallium oxide may be used as the metal oxide 231a and the metal oxide 231c, in the case where the metal oxide 231b is an In—Ga—Zn oxide. The metal oxide 231c may have a stacked-layer structure. For example, a stacked-layer structure of an In—Ga—Zn oxide and a Ga—Zn oxide over the In—Ga—Zn oxide or a stacked-layer structure of an In—Ga—Zn oxide and gallium oxide over the In—Ga—Zn oxide can be employed. In other words, the metal oxide 231c may have a stacked-layer structure of an In—Ga—Zn oxide and an oxide that does not contain In.

Specifically, as the metal oxide 231a, a metal oxide with In:Ga:Zn=1:3:4 [atomic ratio] or 1:1:0.5 [atomic ratio] can be used. As the metal oxide 231b, a metal oxide with In:Ga:Zn=4:2:3 [atomic ratio] or 3:1:2 [atomic ratio] can be used. As the metal oxide 231c, a metal oxide with In:Ga:Zn=1:3:4 [atomic ratio], In:Ga:Zn=4:2:3 [atomic ratio], Ga:Zn=2:1 [atomic ratio], or Ga:Zn=2:5 [atomic ratio] can be used. Specific examples of a stacked-layer structure of the metal oxide 231c include a stacked-layer structure of a layer with In:Ga:Zn=4:2:3 [atomic ratio] and a layer with Ga:Zn=2:1 [atomic ratio], a stacked-layer structure of a layer with In:Ga:Zn=4:2:3 [atomic ratio] and a layer with Ga:Zn=2:5 [atomic ratio], and a stacked-layer structure of a layer with In:Ga:Zn=4:2:3 [atomic ratio] and a layer of gallium oxide.

At this time, the metal oxide 231b serves as a main carrier path. When the metal oxide 231a and the metal oxide 231c have the above structure, the density of defect states at the interface between the metal oxide 231a and the metal oxide 231b and the interface between the metal oxide 231b and the metal oxide 231c can be made low. This reduces the influence of interface scattering on carrier conduction, and the transistor 200 can have a high on-state current and high frequency characteristics. Note that in the case where the metal oxide 231c has a stacked-layer structure, not only the effect of reducing the density of defect states at the interface between the metal oxide 231b and the metal oxide 231c, but also the effect of inhibiting diffusion of the constituent element contained in the metal oxide 231c to the insulator 250 side can be expected. Specifically, the metal oxide 231c has a stacked-layer structure in which an oxide not containing In is positioned in the upper layer of the stacked-layer structure, whereby the diffusion of In to the insulator 250 side can be inhibited. Since the insulator 250 functions as a gate insulator, the transistor has defects in characteristics when In diffuses. Thus, the metal oxide 231c having a stacked-layer structure allows a highly reliable display device to be provided.

The conductor 242a and the conductor 242b functioning as the source electrode and the drain electrode is provided over the metal oxide 231b. For the conductor 242a and the conductor 242b, it is preferable to use a metal element selected from aluminum, chromium, copper, silver, gold, platinum, tantalum, nickel, titanium, molybdenum, tungsten, hafnium, vanadium, niobium, manganese, magnesium, zirconium, beryllium, indium, ruthenium, iridium, strontium, and lanthanum; an alloy containing any of the above metal elements; or an alloy containing a combination of the above metal elements. For example, it is preferable to use tantalum nitride, titanium nitride, tungsten, a nitride containing titanium and aluminum, a nitride containing tantalum and aluminum, ruthenium oxide, ruthenium nitride, an oxide containing strontium and ruthenium, or an oxide containing lanthanum and nickel. Tantalum nitride, titanium nitride, a nitride containing titanium and aluminum, a nitride containing tantalum and aluminum, ruthenium oxide, ruthenium nitride, an oxide containing strontium and ruthenium, and an oxide containing lanthanum and nickel are preferable because they are oxidation-resistant conductive materials or materials that maintain their conductivity even after absorbing oxygen.

When the conductor 242a and the conductor 242b are provided to be in contact with the metal oxide 231, the oxygen concentration of the oxide 231 in the vicinity of the conductor 242a and the conductor 242b sometimes decreases. In addition, a metal compound layer that contains the metal contained in the conductor 242a and the conductor 242b and the component of the metal oxide 231 is sometimes formed in the metal oxide 231 in the vicinity of the conductor 242a and the conductor 242b. In such cases, the carrier concentration of a region in the metal oxide 231 in the vicinity of the conductor 242a and the conductor 242b increases, and the region becomes a low-resistance region.

Here, the region between the conductor 242a and the conductor 242b is formed to overlap with the opening of the insulator 279. Accordingly, the conductor 260 can be placed in a self-aligned manner between the conductor 242a and the conductor 242b.

The insulator 250 functions as a gate insulator. The insulator 250 is preferably placed in contact with the top surface of the metal oxide 231c. For the insulator 250, silicon oxide, silicon oxynitride, silicon nitride oxide, silicon nitride, silicon oxide to which fluorine is added, silicon oxide to which carbon is added, silicon oxide to which carbon and nitrogen are added, or porous silicon oxide can be used. In particular, silicon oxide and silicon oxynitride, which are thermally stable, are preferable.

As in the insulator 224, the concentration of impurities typified by water or hydrogen in the insulator 250 is preferably reduced. The thickness of the insulator 250 is preferably greater than or equal to 1 nm and less than or equal to 20 nm.

A metal oxide may be provided between the insulator 250 and the conductor 260. The metal oxide preferably inhibits oxygen diffusion from the insulator 250 into the conductor 260. Accordingly, oxidation of the conductor 260 due to oxygen in the insulator 250 can be inhibited.

The metal oxide functions as part of the gate insulator in some cases. Therefore, when silicon oxide or silicon oxynitride is used for the insulator 250, a metal oxide that is a high-k material with a high dielectric constant is preferably used as the metal oxide. When the gate insulator has a stacked-layer structure of the insulator 250 and the metal oxide, the stacked-layer structure can be thermally stable and have a high dielectric constant. Accordingly, a gate potential applied during operation of the transistor can be reduced while the physical thickness of the gate insulator is maintained. In addition, the equivalent oxide thickness (EOT) of the insulator functioning as the gate insulator can be reduced.

Specifically, a metal oxide containing one kind or two or more kinds selected from hafnium, aluminum, gallium, yttrium, zirconium, tungsten, titanium, tantalum, nickel, germanium, and magnesium can be used. It is particularly preferable to use an insulator containing an oxide of one or both of aluminum and hafnium, such as aluminum oxide, hafnium oxide, or an oxide containing aluminum and hafnium (hafnium aluminate).

Although the conductor 260 is illustrated to have a two-layer structure in FIG. 18, the conductor 260 may have a single-layer structure or a stacked-layer structure of three or more layers.

The conductor 260a is preferably formed using the aforementioned conductor having a function of inhibiting diffusion of impurities typified by a hydrogen atom, a hydrogen molecule, a water molecule, a nitrogen atom, a nitrogen molecule, a nitrogen oxide molecule (N₂O, NO, or NO₂), and a copper atom. Alternatively, it is preferable to use a conductive material having a function of inhibiting diffusion of oxygen (an oxygen atom or an oxygen molecule).

When the conductor 260a has a function of inhibiting diffusion of oxygen, the conductivity of the conductor 260b can be inhibited from being lowered by oxidation due to oxygen contained in the insulator 250. As a conductive material having a function of inhibiting oxygen diffusion, for example, tantalum, tantalum nitride, ruthenium, or ruthenium oxide is preferably used.

Moreover, a conductive material containing tungsten, copper, or aluminum as its main component is preferably used for the conductor 260b. The conductor 260 also functions as a wiring and thus is preferably formed using a conductor having high conductivity. For example, a conductive material containing tungsten, copper, or aluminum as its main component can be used. The conductor 260b may have a stacked-layer structure, for example, a stacked-layer structure of titanium or titanium nitride and the above conductive material.

As illustrated in FIG. 18A and FIG. 18C, the side surface of the metal oxide 231 is positioned to be covered with the conductor 260 in a region where the metal oxide 231b does not overlap with the conductor 242a and the conductor 242b, that is, the channel formation region of the metal oxide 231. Accordingly, electric fields of the conductor 260 functioning as the first gate electrode are likely to act on the side surface of the metal oxide 231. Thus, the on-state current of the transistor 200 can be increased and the frequency characteristics can be improved.

The insulator 254, like the insulator 214, preferably functions as a barrier insulating film that inhibits the entry of impurities typified by water or hydrogen into the transistor 200 from the insulator 279 side. The insulator 254 preferably has a lower hydrogen permeability than the insulator 224, for example. Furthermore, as illustrated in FIG. 18B and FIG. 18C, the insulator 254 is preferably in contact with the side surface of the metal oxide 231c, the top surface and the side surface of the conductor 242a, the top surface and the side surface of the conductor 242b, side surfaces of the metal oxide 231a and the metal oxide 231b, and the top surface of the insulator 224. Such a structure can inhibit the entry of hydrogen contained in the insulator 279 into the metal oxide 231 through the top surfaces or side surfaces of the conductor 242a, the conductor 242b, the metal oxide 231a, the metal oxide 231b, and the insulator 224.

Furthermore, it is preferable that the insulator 254 have a function of inhibiting diffusion of oxygen (an oxygen atom or an oxygen molecule) (it is preferable that the oxygen be less likely to pass through the insulator 254). For example, the insulator 254 preferably has a lower oxygen permeability than the insulator 279 or the insulator 224.

The insulator 254 is preferably formed by a sputtering method. When the insulator 254 is formed by a sputtering method in an oxygen-containing atmosphere, oxygen can be added to the vicinity of a region of the insulator 224 that is in contact with the insulator 254. Thus, oxygen can be supplied from the region to the metal oxide 231 through the insulator 224. Here, with the insulator 254 having a function of inhibiting upward diffusion of oxygen, oxygen can be prevented from diffusing from the metal oxide 231 into the insulator 279. Moreover, with the insulator 222 having a function of inhibiting downward diffusion of oxygen, oxygen diffusion from the metal oxide 231 to the substrate side can be prevented. In the above manner, oxygen is supplied to the channel formation region of the metal oxide 231. Accordingly, oxygen vacancies in the metal oxide 231 can be reduced, so that the transistor can be prevented from having normally-on characteristics.

As the insulator 254, an insulator containing an oxide of one or both of aluminum and hafnium is preferably formed, for example. Note that as the insulator containing an oxide of one or both of aluminum and hafnium, aluminum oxide, hafnium oxide, or an oxide containing aluminum and hafnium (hafnium aluminate) is preferably used.

The insulator 224, the insulator 250, and the metal oxide 231 are covered with the insulator 254 having a barrier property against hydrogen, whereby the insulator 279 is isolated from the insulator 224, the metal oxide 231, and the insulator 250 by the insulator 254. This can inhibit the entry of impurities typified by hydrogen from outside of the transistor 200, resulting in favorable electrical characteristics and high reliability of the transistor 200.

The insulator 279 is provided over the insulator 224, the metal oxide 231, the conductor 242a, and the conductor 242b with the insulator 254 therebetween. The insulator 279 preferably includes, for example, silicon oxide, silicon oxynitride, silicon nitride oxide, silicon oxide to which fluorine is added, silicon oxide to which carbon is added, silicon oxide to which carbon and nitrogen are added, or porous silicon oxide. In particular, silicon oxide and silicon oxynitride are preferable because they are thermally stable. In particular, materials such as silicon oxide, silicon oxynitride, and porous silicon oxide are preferably used, in which case a region containing oxygen to be released by heating can be easily formed.

The concentration of impurities typified by water or hydrogen in the insulator 279 is preferably reduced. In addition, a top surface of the insulator 279 may be planarized.

Like the insulator 214, the insulator 274 preferably functions as a barrier insulating film that inhibits the entry of impurities typified by water or hydrogen into the insulator 279 from the above. As the insulator 274, for example, the insulator that can be used as the insulator 214 and the insulator 254 can be used.

The insulator 281 functioning as an interlayer film is preferably provided over the insulator 274. As in the insulator 224, the concentration of impurities typified by water or hydrogen in the insulator 281 is preferably reduced.

The conductor 245a and the conductor 245b are placed in openings formed in the insulator 281, the insulator 274, the insulator 279, and the insulator 254. The conductor 245a and the conductor 245b are provided to face each other with the conductor 260 interposed therebetween. Note that top surfaces of the conductor 245a and the conductor 245b may be on the same plane as the top surface of the insulator 281.

The insulator 241a is provided in contact with the inner wall of the opening in the insulator 281, the insulator 274, the insulator 279, and the insulator 254, and the first conductor of the conductor 245a is formed in contact with the side surface of the insulator 241a. The conductor 242a is positioned on at least part of the bottom portion of the opening, and the conductor 245a is in contact with the conductor 242a. Similarly, the insulator 241b is provided in contact with the inner wall of the opening in the insulator 281, the insulator 274, the insulator 279, and the insulator 254, and the first conductor of the conductor 245b is formed in contact with the side surface of the insulator 241b. The conductor 242b is positioned on at least part of the bottom portion of the opening, and the conductor 245b is in contact with the conductor 242b.

The conductor 245a and the conductor 245b are preferably formed using a conductive material containing tungsten, copper, or aluminum as its main component. The conductor 245a and the conductor 245b may have a stacked-layer structure.

In the case where each of the conductor 242a and the conductor 242b has a stacked-layer structure, the aforementioned conductor having a function of inhibiting diffusion of impurities typified by water or hydrogen is preferably used as the conductor in contact with the metal oxide 231a, the metal oxide 231b, the conductor 242a, the conductor 242b, the insulator 254, the insulator 279, the insulator 274, and the insulator 281. For example, tantalum, tantalum nitride, titanium, titanium nitride, ruthenium, or ruthenium oxide is preferably used. The conductive material having a function of inhibiting diffusion of impurities typified by water or hydrogen can be used as a single layer or stacked layers. The use of the conductive material can inhibit oxygen added to the insulator 279 from being absorbed by the conductor 245a and the conductor 245b. Moreover, impurities typified by water or hydrogen can be inhibited from entering the metal oxide 231 through the conductor 245a and the conductor 245b from a layer above the insulator 281.

As the insulator 241a and the insulator 241b, for example, the insulator that can be used as the insulator 254 can be used. Since the insulator 241a and the insulator 241b are provided in contact with the insulator 254, impurities typified by water or hydrogen in the insulator 279 can be inhibited from entering the metal oxide 231 through the conductor 245a and the conductor 245b. Furthermore, oxygen contained in the insulator 279 can be inhibited from being absorbed by the conductor 245a and the conductor 245b.

Although not illustrated, a conductor functioning as a wiring may be placed in contact with a top surface of the conductor 245a and a top surface of the conductor 245b. For the conductor functioning as a wiring, a conductive material containing tungsten, copper, or aluminum as its main component is preferably used. Furthermore, the conductor may have a stacked-layer structure and may be a stack of titanium or a titanium nitride and the above conductive material, for example. Note that the conductor may be formed to be embedded in an opening provided in an insulator.

### <Materials for Transistor>

Materials that can be used for the transistor will be described.

### [Substrate]

As a substrate where the transistor 200 is formed, an insulator substrate, a semiconductor substrate, or a conductor substrate is used, for example. Examples of the insulator substrate include a glass substrate, a quartz substrate, a sapphire substrate, a stabilized zirconia substrate (e.g., an yttria-stabilized zirconia substrate), and a resin substrate. Examples of the semiconductor substrate include a semiconductor substrate of silicon or germanium and a compound semiconductor substrate of silicon carbide, silicon germanium, gallium arsenide, indium phosphide, zinc oxide, or gallium oxide. Another example is a semiconductor substrate in which an insulator region is included in the semiconductor substrate, e.g., an SOI (Silicon On Insulator) substrate. Examples of the conductor substrate include a graphite substrate, a metal substrate, an alloy substrate, and a conductive resin substrate. Other examples include a substrate including a metal nitride and a substrate including a metal oxide. Other examples include an insulator substrate provided with a conductor or a semiconductor, a semiconductor substrate provided with a conductor or an insulator, and a conductor substrate provided with a semiconductor or an insulator. Alternatively, these substrates provided with elements may be used. Examples of the elements provided for the substrates include a capacitor, a resistor, a switching element, a light-emitting element, and a memory element.

### [Insulator]

Examples of an insulator include an oxide, a nitride, an oxynitride, a nitride oxide, a metal oxide, a metal oxynitride, and a metal nitride oxide, each of which has an insulating property.

With further miniaturization and higher integration of a transistor, for example, a problem such as generation of leakage current may arise because of a thinned gate insulator. When a high-k material is used for the insulator functioning as a gate insulator, the voltage at the time of operation of the transistor can be reduced while the physical thickness is maintained. By contrast, when a material with a low dielectric constant is used for the insulator functioning as an interlayer film, parasitic capacitance generated between wirings can be reduced. Thus, a material is preferably selected depending on the function of an insulator.

Examples of the insulator having a high dielectric constant include gallium oxide, hafnium oxide, zirconium oxide, an oxide containing aluminum and hafnium, an oxynitride containing aluminum and hafnium, an oxide containing silicon and hafnium, an oxynitride containing silicon and hafnium, and a nitride containing silicon and hafnium.

Examples of the insulator having a low dielectric constant include silicon oxide, silicon oxynitride, silicon nitride oxide, silicon nitride, silicon oxide to which fluorine is added, silicon oxide to which carbon is added, silicon oxide to which carbon and nitrogen are added, porous silicon oxide, and a resin.

When a transistor including an oxide semiconductor is surrounded by insulators having a function of inhibiting the passage of oxygen and impurities typified by hydrogen (the insulator 214, the insulator 222, the insulator 254, and the insulator 274), the electrical characteristics of the transistor can be stable. An insulator having a function of inhibiting the passage of oxygen and impurities typified by hydrogen can be formed to have a single layer or a stacked layer including an insulator containing, for example, boron, carbon, nitrogen, oxygen, fluorine, magnesium, aluminum, silicon, phosphorus, chlorine, argon, gallium, germanium, yttrium, zirconium, lanthanum, neodymium, hafnium, or tantalum. Specifically, as the insulator having a function of inhibiting the passage of oxygen and impurities typified by hydrogen, a metal oxide typified by aluminum oxide, magnesium oxide, gallium oxide, germanium oxide, yttrium oxide, zirconium oxide, lanthanum oxide, neodymium oxide, hafnium oxide, or tantalum oxide or a metal nitride typified by aluminum nitride, aluminum titanium nitride, titanium nitride, silicon nitride oxide, or silicon nitride can be used.

An insulator functioning as a gate insulator is preferably an insulator including a region containing oxygen to be released by heating. For example, when a structure is employed in which silicon oxide or silicon oxynitride that includes a region containing oxygen to be released by heating is provided in contact with the metal oxide 231, oxygen vacancies included in the metal oxide 231 can be compensated.

### [Conductor]

For a conductor, it is preferable to use a metal element selected from aluminum, chromium, copper, silver, gold, platinum, tantalum, nickel, titanium, molybdenum, tungsten, hafnium, vanadium, niobium, manganese, magnesium, zirconium, beryllium, indium, ruthenium, iridium, strontium, and lanthanum; an alloy containing any of the above metal elements; or an alloy containing a combination of the above metal elements. For example, it is preferable to use tantalum nitride, titanium nitride, tungsten, a nitride containing titanium and aluminum, a nitride containing tantalum and aluminum, ruthenium oxide, ruthenium nitride, an oxide containing strontium and ruthenium, and an oxide containing lanthanum and nickel. Tantalum nitride, titanium nitride, a nitride containing titanium and aluminum, a nitride containing tantalum and aluminum, ruthenium oxide, ruthenium nitride, an oxide containing strontium and ruthenium, and an oxide containing lanthanum and nickel are preferable because they are oxidation-resistant conductive materials or materials that maintain their conductivity even after absorbing oxygen. A semiconductor having high electrical conductivity, typified by polycrystalline silicon containing an impurity element typified by phosphorus, or silicide such as nickel silicide may be used.

A plurality of conductors formed using any of the above materials may be stacked. For example, a stacked-layer structure combining a material containing the above metal element and a conductive material containing oxygen may be employed. In addition, a stacked-layer structure combining a material containing the above metal element and a conductive material containing nitrogen may be employed. Furthermore, a stacked-layer structure combining a material containing the above metal element, a conductive material containing oxygen, and a conductive material containing nitrogen may be employed.

In the case where a metal oxide is used for the channel formation region of the transistor, the conductor functioning as the gate electrode preferably employs a stacked-layer structure combining a material containing the above metal element and a conductive material containing oxygen. In that case, the conductive material containing oxygen is preferably provided on the channel formation region side. When the conductive material containing oxygen is provided on the channel formation region side, oxygen released from the conductive material is easily supplied to the channel formation region.

It is particularly preferable to use, for the conductor functioning as the gate electrode, a conductive material containing oxygen and a metal element contained in the metal oxide where the channel is formed. A conductive material containing the above metal element and nitrogen may be used. For example, a conductive material containing nitrogen, such as titanium nitride or tantalum nitride, may be used. Indium tin oxide, indium oxide containing tungsten oxide, indium zinc oxide containing tungsten oxide, indium oxide containing titanium oxide, indium tin oxide containing titanium oxide, indium zinc oxide, or indium tin oxide to which silicon is added may be used. Indium gallium zinc oxide containing nitrogen may be used. With the use of such a material, hydrogen contained in the metal oxide where the channel is formed can be captured in some cases. Alternatively, hydrogen entering from an external insulator can be captured in some cases.

At least part of this embodiment can be implemented in combination with the other embodiments described in this specification as appropriate.

The structures described in this embodiment can be used in an appropriate combination with the structures described in the other embodiments.

### Embodiment 6

Described in this embodiment is a metal oxide (hereinafter also referred to as an oxide semiconductor) that can be used in an OS transistor described in the above embodiment.

### <Classification of Crystal Structure>

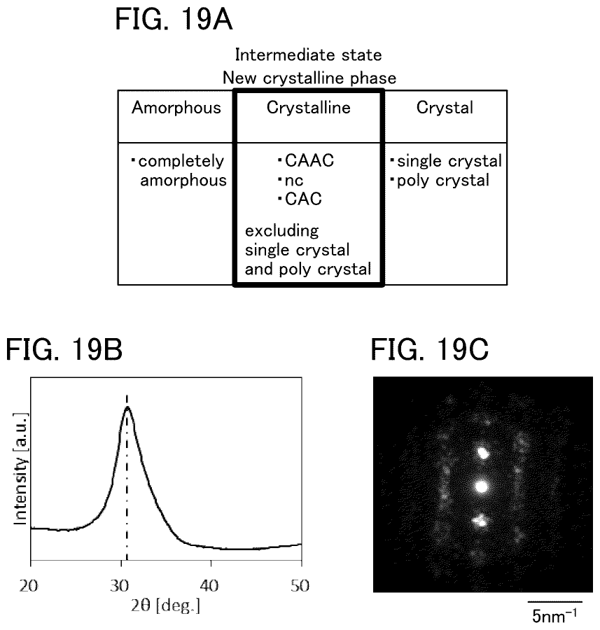

First, the classification of crystal structures of an oxide semiconductor is described with reference to FIG. 19A. FIG. 19A is a diagram showing classification of crystal structures of an oxide semiconductor, typically IGZO (a metal oxide containing In, Ga, and Zn).

As shown in FIG. 19A, an oxide semiconductor is roughly classified into “Amorphous”, “Crystalline”, and “Crystal”. “Amorphous” includes completely amorphous. The term “Crystalline” includes CAAC (c-axis-aligned crystalline), nc (nanocrystalline), and CAC (cloud-aligned composite) (excluding single crystal and poly crystal). Note that the term “Crystalline” excludes single crystal, poly crystal, and completely amorphous. Moreover, the term “Crystal” includes single crystal and poly crystal.

Note that the structures in the thick frame in FIG. 19A are in an intermediate state between “Amorphous” and “Crystal”, and belong to a new crystalline phase. That is, these structures are completely different from “Crystal” and “Amorphous”, which is energetically unstable.

A crystal structure of a film or a substrate can be evaluated with an X-Ray Diffraction (XRD) spectrum. FIG. 19B shows an XRD spectrum, which is obtained using GIXD (Grazing-Incidence XRD) measurement, of a CAAC-IGZO film classified into “Crystalline”. Note that a GIXD method is also referred to as a thin film method or a Seemann—Bohlin method. The XRD spectrum that is shown in FIG. 19B and obtained by GIXD measurement is hereinafter simply referred to as an XRD spectrum. In FIG. 19B, the vertical axis represents Intensity and the horizontal axis represents 20. The CAAC-IGZO film in FIG. 19B has a composition in the vicinity of In:Ga:Zn=4:2:3 [atomic ratio]. The CAAC-IGZO film in FIG. 19B has a thickness of 500 nm.

As shown in FIG. 19B, a clear peak indicating crystallinity is detected in the XRD spectrum of the CAAC-IGZO film. Specifically, a peak indicating c-axis alignment is detected at 20 of around 31° in the XRD spectrum of the CAAC-IGZO film. As shown in FIG. 19B, the peak at 20 of around 31° is asymmetric with respect to the axis of the angle at which the peak intensity is detected.

A crystal structure of a film or a substrate can also be evaluated with a diffraction pattern obtained by a nanobeam electron diffraction (NBED) method (such a pattern is also referred to as a nanobeam electron diffraction pattern). FIG. 19C shows a diffraction pattern of a CAAC-IGZO film. FIG. 19C shows a diffraction pattern obtained with the NBED method in which an electron beam is incident in the direction parallel to the substrate. The CAAC-IGZO film in FIG. 19C has a composition in the vicinity of In:Ga:Zn=4:2:3 [atomic ratio]. In the nanobeam electron diffraction method, electron diffraction is performed with a probe diameter of 1 nm.

As shown in FIG. 19C, a plurality of spots indicating c-axis alignment are observed in the diffraction pattern of the CAAC-IGZO film.

### [Structure of Oxide Semiconductor]

Oxide semiconductors might be classified in a manner different from that in FIG. 19A when classified in terms of the crystal structure. Oxide semiconductors are classified into a single crystal oxide semiconductor and a non-single-crystal oxide semiconductor, for example. Examples of the non-single-crystal oxide semiconductor include the above-described CAAC-OS and nc-OS. Other examples of the non-single-crystal oxide semiconductor include a polycrystalline oxide semiconductor, an amorphous-like oxide semiconductor (a-like OS), and an amorphous oxide semiconductor.

Here, the above-described CAAC-OS, nc-OS, and a-like OS are described in detail.

### [CAAC-OS]

The CAAC-OS is an oxide semiconductor that has a plurality of crystal regions each of which has c-axis alignment in a particular direction. Note that the particular direction refers to the film thickness direction of a CAAC-OS film, the normal direction of the surface where the CAAC-OS film is formed, or the normal direction of the surface of the CAAC-OS film. The crystal region refers to a region having a periodic atomic arrangement. When an atomic arrangement is regarded as a lattice arrangement, the crystal region also refers to a region with a uniform lattice arrangement. The CAAC-OS has a region where a plurality of crystal regions are connected in the a-b plane direction, and the region has distortion in some cases. Note that the distortion refers to a portion where the direction of a lattice arrangement changes between a region with a uniform lattice arrangement and another region with a uniform lattice arrangement in a region where a plurality of crystal regions are connected. That is, the CAAC-OS is an oxide semiconductor having c-axis alignment and having no clear alignment in the a-b plane direction.

Note that each of the plurality of crystal regions is formed of one or more fine crystals (crystals each of which has a maximum diameter of less than 10 nm). In the case where the crystal region is formed of one fine crystal, the maximum diameter of the crystal region is less than 10 nm. In the case where the crystal region is formed of a large number of fine crystals, the size of the crystal region may be approximately several tens of nanometers.

In the case of an In-M-Zn oxide (the element M is one or more of aluminum, gallium, yttrium, tin, titanium, and the like), the CAAC-OS tends to have a layered crystal structure (also referred to as a stacked-layer structure) in which a layer containing indium (In) and oxygen (hereinafter, an In layer) and a layer containing the element M, zinc (Zn), and oxygen (hereinafter, an (M,Zn) layer) are stacked. Indium and the element M can be replaced with each other. Therefore, indium may be contained in the (M,Zn) layer. In addition, the element M may be contained in the In layer. Note that Zn may be contained in the In layer. Such a layered structure is observed as a lattice image in a high-resolution TEM image, for example.

When the CAAC-OS film is subjected to structural analysis by out-of-plane XRD measurement with an XRD apparatus using θ/2θ scanning, for example, a peak indicating c-axis alignment is detected at 2θ of 31° or around 31°. Note that the position of the peak indicating c-axis alignment (the value of 2θ) may change depending on the kind or composition of the metal element contained in the CAAC-OS.

For example, a plurality of bright spots are observed in the electron diffraction pattern of the CAAC-OS film. Note that one spot and another spot are observed point-symmetrically with a spot of the incident electron beam passing through a sample (also referred to as a direct spot) as the symmetric center.

When the crystal region is observed from the particular direction, a lattice arrangement in the crystal region is basically a hexagonal lattice arrangement; however, a unit lattice is not always a regular hexagon and is a non-regular hexagon in some cases. A pentagonal lattice arrangement or a heptagonal lattice arrangement is included in the distortion in some cases. Note that a clear crystal grain boundary (grain boundary) cannot be observed even in the vicinity of the distortion in the CAAC-OS. That is, formation of a crystal grain boundary is inhibited by the distortion of lattice arrangement. This is probably because the CAAC-OS can tolerate distortion owing to a low density of arrangement of oxygen atoms in the a-b plane direction or an interatomic bond distance changed by substitution of a metal atom.

A crystal structure in which a clear crystal grain boundary is observed is what is called polycrystal. It is highly probable that the crystal grain boundary becomes a recombination center and traps carriers and thus decreases the on-state current or field-effect mobility of a transistor. Thus, the CAAC-OS in which no clear crystal grain boundary is observed is one of crystalline oxides having a crystal structure suitable for a semiconductor layer of a transistor. Note that Zn is preferably contained to form the CAAC-OS. For example, an In—Zn oxide and an In—Ga—Zn oxide are suitable because they can inhibit generation of a grain boundary as compared with an In oxide.

The CAAC-OS is an oxide semiconductor with high crystallinity in which no clear crystal grain boundary is observed. Thus, in the CAAC-OS, reduction in electron mobility due to the crystal grain boundary is less likely to occur. Moreover, since the crystallinity of an oxide semiconductor might be decreased by entry of impurities and/or formation of defects, the CAAC-OS can be regarded as an oxide semiconductor that has small amounts of impurities and defects (oxygen vacancies). Thus, an oxide semiconductor including the CAAC-OS is physically stable. Therefore, the oxide semiconductor including the CAAC-OS is resistant to heat and has high reliability. In addition, the CAAC-OS is stable with respect to high temperatures in the manufacturing process (what is called thermal budget). Accordingly, the use of the CAAC-OS for the OS transistor can extend the degree of freedom of the manufacturing process.

[nc-OS]

In the nc-OS, a microscopic region (e.g., a region with a size greater than or equal to 1 nm and less than or equal to 10 nm, in particular, a region with a size greater than or equal to 1 nm and less than or equal to 3 nm) has a periodic atomic arrangement. In other words, the nc-OS includes a fine crystal. Note that the size of the fine crystal is, for example, greater than or equal to 1 nm and less than or equal to 10 nm, particularly greater than or equal to 1 nm and less than or equal to 3 nm; thus, the fine crystal is also referred to as a nanocrystal. Furthermore, there is no regularity of crystal orientation between different nanocrystals in the nc-OS. Thus, the orientation in the whole film is not observed. Accordingly, the nc-OS cannot be distinguished from an a-like OS or an amorphous oxide semiconductor by some analysis methods. For example, when an nc-OS film is subjected to structural analysis using out-of-plane XRD measurement with an XRD apparatus using θ/2θ scanning, a peak indicating crystallinity is not detected. Furthermore, a diffraction pattern like a halo pattern is observed when the nc-OS film is subjected to electron diffraction (also referred to as selected-area electron diffraction) using an electron beam with a probe diameter greater than the diameter of a nanocrystal (e.g., greater than or equal to 50 nm). Meanwhile, in some cases, a plurality of spots in a ring-like region with a direct spot as the center are observed in the obtained electron diffraction pattern when the nc-OS film is subjected to electron diffraction (also referred to as nanobeam electron diffraction) using an electron beam with a probe diameter nearly equal to or less than the diameter of a nanocrystal (e.g., greater than or equal to 1 nm and less than or equal to 30 nm).

[a-like OS]

The a-like OS is an oxide semiconductor having a structure between those of the nc-OS and the amorphous oxide semiconductor. The a-like OS includes a void or a low-density region. That is, the a-like OS has low crystallinity as compared with the nc-OS and the CAAC-OS. Moreover, the a-like OS has higher hydrogen concentration in the film than the nc-OS and the CAAC-OS.

### [Structure of Oxide Semiconductor]

Next, the above-described CAC-OS is described in detail. Note that the CAC-OS relates to the material composition.

### [CAC-OS]

The CAC-OS refers to one composition of a material in which elements constituting a metal oxide are unevenly distributed with a size greater than or equal to 0.5 nm and less than or equal to 10 nm, preferably greater than or equal to 1 nm and less than or equal to 3 nm, or a similar size, for example. Note that a state in which one or more metal elements are unevenly distributed and regions including the metal element(s) are mixed with a size greater than or equal to 0.5 nm and less than or equal to 10 nm, preferably greater than or equal to 1 nm and less than or equal to 3 nm, or a similar size in a metal oxide is hereinafter referred to as a mosaic pattern or a patch-like pattern.

In addition, the CAC-OS has a composition in which materials are separated into a first region and a second region to form a mosaic pattern, and the first regions are distributed in the film (this composition is hereinafter also referred to as a cloud-like composition). That is, the CAC-OS is a composite metal oxide having a composition in which the first regions and the second regions are mixed.

Here, the atomic ratios of In, Ga, and Zn to the metal elements contained in the CAC-OS in an In—Ga—Zn oxide are denoted by [In], [Ga], and [Zn], respectively. For example, the first region in the CAC-OS in the In—Ga—Zn oxide has [In] higher than [In] in the composition of the CAC-OS film. Moreover, the second region has [Ga] higher than [Ga] in the composition of the CAC-OS film. Alternatively, for example, the first region has higher [In] and lower [Ga] than the second region. Moreover, the second region has higher [Ga] and lower [In] than the first region.

Specifically, the first region includes indium oxide or indium zinc oxide as its main component. The second region includes gallium oxide or gallium zinc oxide as its main component. That is, the first region can be referred to as a region containing In as its main component. The second region can be referred to as a region containing Ga as its main component.

Note that a clear boundary between the first region and the second region cannot be observed in some cases.

For example, energy dispersive X-ray spectroscopy (EDX) is used to obtain EDX mapping, and according to the EDX mapping, the CAC-OS in the In—Ga—Zn oxide has a structure in which the region containing In as its main component (the first region) and the region containing Ga as its main component (the second region) are unevenly distributed and mixed.

In the case where the CAC-OS is used for a transistor, a switching function (on/off switching function) can be given to the CAC-OS owing to the complementary action of the conductivity derived from the first region and the insulating property derived from the second region. That is, the CAC-OS has a conducting function in part of the material and has an insulating function in another part of the material; as a whole, the CAC-OS has a function of a semiconductor. Separation of the conducting function and the insulating function can maximize each function. Accordingly, when the CAC-OS is used for a transistor, high on-state current (Ion), high field-effect mobility (μ), and excellent switching operation can be achieved.

An oxide semiconductor has various structures with different properties. Two or more kinds among the amorphous oxide semiconductor, the polycrystalline oxide semiconductor, the a-like OS, the CAC-OS, the nc-OS, and the CAAC-OS may be included in the oxide semiconductor of one embodiment of the present invention.

### <Transistor Including Oxide Semiconductor>

Next, the case where the above oxide semiconductor is used for a transistor is described.

When the above oxide semiconductor is used for a transistor, a transistor with high field-effect mobility can be achieved. In addition, a transistor having high reliability can be achieved.

An oxide semiconductor with a low carrier concentration is preferably used for a transistor. For example, the carrier concentration of an oxide semiconductor is lower than or equal to 1×10¹⁷ cm⁻³, preferably lower than or equal to 1×10¹⁵ cm⁻³, further preferably lower than or equal to 1×10¹³ cm⁻³, still further preferably lower than or equal to 1×10¹¹ cm⁻³, yet further preferably lower than 1×10¹⁰ cm⁻³, and higher than or equal to 1×10⁻⁹ cm⁻³. In order to reduce the carrier concentration in an oxide semiconductor film, the impurity concentration in the oxide semiconductor film is reduced so that the density of defect states can be reduced. In this specification, a state with a low impurity concentration and a low density of defect states is referred to as a highly purified intrinsic or substantially highly purified intrinsic state. Note that an oxide semiconductor having a low carrier concentration may be referred to as a highly purified intrinsic or substantially highly purified intrinsic oxide semiconductor.

A highly purified intrinsic or substantially highly purified intrinsic oxide semiconductor film has a low density of defect states and accordingly has a low density of trap states in some cases.

Electric charge trapped by the trap states in the oxide semiconductor takes a long time to disappear and might behave like fixed electric charge. Thus, a transistor whose channel formation region is formed in an oxide semiconductor with a high density of trap states has unstable electrical characteristics in some cases.

Accordingly, in order to obtain stable electrical characteristics of a transistor, reducing the impurity concentration in an oxide semiconductor is effective. In order to reduce the impurity concentration in the oxide semiconductor, it is preferable that the impurity concentration in an adjacent film be also reduced. Examples of impurities include hydrogen, nitrogen, an alkali metal, an alkaline earth metal, iron, nickel, and silicon.

### <Impurity>

Here, the influence of each impurity in the oxide semiconductor is described.

When silicon and/or carbon, which are each one of Group 14 elements, is contained in the oxide semiconductor, defect states are formed in the oxide semiconductor. Thus, the concentration of silicon and carbon in the oxide semiconductor and the concentration of silicon and carbon in the vicinity of an interface with the oxide semiconductor (the concentrations obtained by SIMS) are each set lower than or equal to 2×10¹⁸ atoms/cm³, preferably lower than or equal to 2×10¹⁷ atoms/cm³.

When the oxide semiconductor contains an alkali metal or an alkaline earth metal, defect states are formed and carriers are generated in some cases. Accordingly, a transistor including an oxide semiconductor that contains an alkali metal or an alkaline earth metal tends to have normally-on characteristics. Thus, the concentration of an alkali metal or an alkaline earth metal in the oxide semiconductor, which is obtained by SIMS, is set lower than or equal to 1×10¹⁸ atoms/cm³, preferably lower than or equal to 2×10¹⁶ atoms/cm³.

Furthermore, when the oxide semiconductor contains nitrogen, the oxide semiconductor easily becomes n-type by generation of electrons serving as carriers and an increase in carrier concentration. As a result, a transistor including an oxide semiconductor containing nitrogen as a semiconductor is likely to have normally-on characteristics. When nitrogen is contained in the oxide semiconductor, a trap state is sometimes formed. This might make the electrical characteristics of the transistor unstable. Therefore, the concentration of nitrogen in the oxide semiconductor, which is obtained by SIMS, is set lower than 5×10¹⁹ atoms/cm³, preferably lower than or equal to 5×10¹⁸ atoms/cm³, further preferably lower than or equal to 1×10¹⁸ atoms/cm³, still further preferably lower than or equal to 5×10¹⁷ atoms/cm³.

Hydrogen contained in the oxide semiconductor reacts with oxygen bonded to a metal atom to be water, and thus forms an oxygen vacancy in some cases. Entry of hydrogen into the oxygen vacancy generates an electron serving as a carrier in some cases. Furthermore, bonding of part of hydrogen to oxygen bonded to a metal atom causes generation of an electron serving as a carrier in some cases. Thus, a transistor using an oxide semiconductor containing hydrogen is likely to have normally-on characteristics. Accordingly, hydrogen in the oxide semiconductor is preferably reduced as much as possible. Specifically, the hydrogen concentration in the oxide semiconductor, which is obtained by SIMS, is set lower than 1×10²⁰ atoms/cm³, preferably lower than 1×10¹⁹ atoms/cm³, further preferably lower than 5×10¹⁸ atoms/cm³, still further preferably lower than 1×10¹⁸ atoms/cm³.

When an oxide semiconductor with sufficiently reduced impurities is used for the channel formation region of the transistor, stable electrical characteristics can be given.

The structures described in this embodiment can be used in an appropriate combination with the structures described in the other embodiments.

### REFERENCE NUMERALS

10: layer, 20: layer, 30: layer, 31: housing, 32: opening portion, 34: fixing unit, 35a: optical component, 35b: optical component, 36a: frame, 36b: frame, 37: battery, 40: sealing substrate, 60: layer, 61: light-emitting element, 61B: light-emitting element, 61G: light-emitting element, 61R: light-emitting element, 61W: light-emitting element, 80: electronic device, 81: user, 91: external camera, 92: battery, 100A: semiconductor device, 100B: semiconductor device, 100C: semiconductor device, 100D: semiconductor device, 100G: semiconductor device, 103: housing, 104: light source, 105: protective member, 130B: light-emitting element, 130G: light-emitting element, 130R: light-emitting element, 200: transistor, 205: conductor, 205a: conductor, 205b: conductor, 205c: conductor, 214: insulator, 216: insulator, 222: insulator, 224: insulator, 231: metal oxide, 231a: metal oxide, 231b: metal oxide, 231c: metal oxide, 241a: insulator, 241b: insulator, 242a: conductor, 242b: conductor, 245a: conductor, 245b: conductor, 250: insulator, 251: insulating layer, 254: insulator, 260: conductor, 260a: conductor, 260b: conductor, 264B: coloring layer, 264G: coloring layer, 264R: coloring layer, 271: protective layer, 272: insulating layer, 273: protective layer, 274: insulator, 275: space, 276: insulating layer, 277: microlens array, 279: insulator, 280: display panel, 280L: display panel, 280R: display panel, 281: insulator, 282: circuit portion, 283: pixel circuit portion, 283a: pixel circuit, 284: pixel portion, 284a: pixel, 284b: pixel portion, 284c: pixel portion, 285: terminal portion, 286: wiring portion, 290: FPC, 291: substrate, 292: substrate, 301a: conductor, 301b: conductor, 311: conductor, 313: conductor, 321: lower electrode, 323: insulator, 325: upper electrode, 331: conductor, 333: conductor, 335: conductor, 341: conductor, 343: conductor, 347: conductor, 348: conductor, 351: conductor, 353: conductor, 355: conductor, 357: conductor, 361: insulator, 363: insulator, 400C: display device, 403: element isolation layer, 405: insulator, 407: insulator, 409: insulator, 411: insulator, 421: insulator, 422: insulator, 423: insulator, 424: insulator, 430a: light-emitting element, 430b: light-emitting element, 430c: light-emitting element, 431: transistor, 441: transistor, 442: transistor, 443: conductor, 445: insulator, 447: semiconductor region, 449a: low-resistance region, 449b: low-resistance region, 451: conductor, 453: conductor, 454: bump, 455: conductor, 456: conductor, 457: adhesive layer, 458: bump, 459: adhesive layer, 461: conductor, 462: conductor, 465: conductor, 701: substrate, 702: substrate, 712: sealant, 716: FPC, 730: insulator, 732: filling layer, 734: insulator, 736: coloring layer, 738: light-blocking layer, 750: transistor, 760: conductor, 772: conductor, 778: component, 780: anisotropic conductor, 786: EL layer, 786a: EL layer, 786b: EL layer, 786B: EL layer, 786G: EL layer, 786R: EL layer, 786W: EL layer, 788: conductor, 790: capacitor, 941: retina, 942: crystalline lens, 943: optic nerve, 944: optic disc, 945: vein, 946: artery, 947: vitreous body, 948: choroid, 950: optical system, 951: emitted light, 951B: light, 951G: light, 9511R: emitted light, 951R: light, 4411: light-emitting layer, 4412: light-emitting layer, 4413: light-emitting layer, 4420: layer, 4420-1: layer, 4420-2: layer, 4430: layer, 4430-1: layer, 4430-2: layer

1. An electronic device worn on a head of a user, comprising:
a display device comprising a transistor;
a light-emitting element; and
a light-receiving element in the same substrate,
wherein the display device is configured to display an image and to capture an image of a fundus of an eye of the user.

2. The electronic device according to claim 1,
wherein the light-emitting element is an organic light-emitting element.

3. The electronic device according to claim 1,
wherein the light-emitting element is an organic light-emitting element emitting infrared light.

4. The electronic device according to claim 1,
wherein a semiconductor layer of the transistor is single crystal silicon.

5. The electronic device according to claim 1,
a semiconductor layer of the transistor is an oxide semiconductor.

6. The electronic device according to claim 1,
wherein diagnosis is made by capturing an image of the eye of the user.

7. An electronic device worn on a head of a user, comprising:
a display device comprising a transistor;
a light-emitting element; and
a light-receiving element over the same substrate,
wherein the display device is configured to display an image and to capture an image of a fundus of an eye of the user.

8. The electronic device according to claim 7,
wherein the light-emitting element is an organic light-emitting element.

9. The electronic device according to claim 7,
wherein the light-emitting element is an organic light-emitting element emitting infrared light.

10. The electronic device according to claim 7,
wherein a semiconductor layer of the transistor is single crystal silicon.

11. The electronic device according to claim 7,
a semiconductor layer of the transistor is an oxide semiconductor.

12. The electronic device according to claim 7,
wherein diagnosis is made by capturing an image of the eye of the user.
